SEQUENCE LISTING (5 sequences)

SEQ ID NO: 1 - protein, 309 aa, Homo Sapiens
MFSPADNIFIILITGEFILGILGNGYIALVNWIDWIKKKKISTVDYILTNLVIARICLISVMVVNGIVIVLNPDVYTKNKQQIVIFTFWTFANYLNMWITTCLNVFYFLKIASSSHPLFLWLKWKIDMVVHWILLGCFAISLLVSLIAAIVLSCDYRFHAIAKHKRNITEMFHVSKIPYFEPLTLFNLFAIVPFIVSLISFFLLVRSLWRHTKQIKLYATGSRDPSTEVHVRAIKTMTSFIFFFFLYYISSILMTFSYLMTKYKLAVEFGEIAAILYPLGHSLILIVLNNKLRQTFVRMLTCRKIACMI

SEQ ID NO: 2 - protein, 319 aa, Homo Sapiens
MITFLPIIFSILIVVIFVIGNFANGFIALVNSIEWVKRQKISFVDQILTALAVSRVGLLWVLLLHWYATQLNPAFYSVEVRITAYNVWAVTNHFSSWLATSLSMFYLLRIANFSNLIFLRIKRRVKSVVLVILLGPLLFLVCHLFVINMDETVWTKEYEGNVTWKIKLRSAMYHSNMTLTMLANFVPLTLTLISFLLLICSLCKHLKKMQLHGKGSQDPSTKVHIKALQTVTSFLLLCAIYFLSMIISVCNLGRLEKQPVFMFCQAIIFSYPSTHPFILILGNKKLKQIFLSVLRHVRYWVKDRSLRLHRFTRGALCVF

SEQ ID NO: 3 - protein, 333 aa, Homo Sapiens [sequence omitted]

SEQ ID NO: 4 - protein, 299 aa, Homo Sapiens
MMCFLLIISSILVVFAFVLGNVANGFIALVNIIDWVNTRKISSAEQILTALVVSRIGLLWVMLFLWYATVFNSALYGLEVRIVASNAWAVTNHFSMWLAASLSIFCLLKIANFSNLISLHLKKRIKSVVLVILLGPLVFLICNLAVITMDERVWTKEYEGNVTWKIKLRNAIHLSSLTVTTLANLIPFTLSLICFLLLICSLCKHLKKMRLHSKGSQDPSTKVHIKALQTVTSFLMLFAIYFLCIITSTWNLRTQQSKLVLLLCQTVAIMYPSFHSFILIMGSRKLKQTFLSVLWQMTR

SEQ ID NO: 5 - protein, 309 aa, Homo Sapiens
MITFLPIIFSILIVVTFVIGNFANGFIALVNSIEWFKRQKISFADQILTALAVSRVGLLWVLVLNWYATELNPAFNSIEVRITAYNVWAVINHFSNWLATSLSIFYLLKIANFSNLIFLHLKRRVKSVVLVILLGPLLFLVCHLFVINMNQIIWTKEYEGNMTWKIKLRSAMYLSNTTVTILANLVPFTLTLISFLLLICSLCKHLKKMQLHGKGSQDPSMKVHIKALQTVTSFLLLCAIYFLSIIMSVWSFESLENKPVFMFCEAIAFSYPSTHPFILIWGNKKLKQTFLSVLWHVRYWVKGEKPSSS

(19) United States
(12) Patent Application Publication
(10) Pub. No.: US 2022/0236254 A1
(43) Pub. Date: Jul. 28, 2022

(54) BITTER TASTE RECEPTOR BLOCKERS AND METHODS OF THEIR IDENTIFICATION

(71) Applicant: Firmenich Incorporated, Plainsboro, NJ (US)

(72) Inventors: Guy Servant, San Diego, CA (US); Mark Williams, San Diego, CA (US); Lan Zhang, San Diego, CA (US)

(21) Appl. No.: 17/630,757

(22) PCT Filed: Sep. 4, 2020
(86) PCT No.: PCT/US2020/049542

§ 371 Date: Jan. 27, 2022

Related U.S. Application Data
(60) Provisional application No. 62/897,844, filed on Sep. 9, 2019.

(51) Int. Cl. G01N 33/50; G01N 33/566; G01N 33/68

(52) U.S. Cl. CPC G01N 33/5008; G01N 2333/726; G01N 33/68; G01N 33/566

(57) ABSTRACT

The present disclosure generally provides methods for identifying compounds that block bitter receptors, particularly the bitter receptors activated by certain compounds commonly present in Citrus extracts. The disclosure also provides compositions comprising such compounds and methods of their use.

### CROSS-REFERENCE TO RELATED APPLICATIONS

The present application claims the benefit of priority of United States Provisional Application Nos. 62/897,844, filed Sep. 9, 2019, and 63/010,655, filed Apr. 15, 2020, and European Patent App. No. 19216401.0, filed Dec. 16, 2019, each of which is incorporated herein by reference as though set forth herein in their entireties.

### REFERENCE TO SEQUENCE LISTING IN ELECTRONIC FORMAT

This application is filed with an electronic sequence listing entitled SNMX054WOSEQLIST.txt, created on Sep. 4, 2020, which is 13,621 bytes in size. The information in the electronic sequence listing is hereby incorporated by reference in its entirety.

### TECHNICAL FIELD

The present disclosure generally provides methods for identifying compounds that block bitter receptors, particularly the bitter receptors activated by certain compounds commonly present in Citrus extracts. The disclosure also provides compositions comprising such compounds and methods of their use.

### DESCRIPTION OF RELATED ART

The taste system provides sensory information about the chemical composition of the external world. Taste transduction is one of the more sophisticated forms of chemically triggered sensation in animals. Signaling of taste is found throughout the animal kingdom, from simple metazoans to the most complex of vertebrates. Mammals are believed to have five basic taste modalities: sweet, bitter, sour, salty, and umami.

Bitterness is one of the most sensitive of these five taste modalities, and people generally perceive bitterness as unpleasant, sharp, and disagreeable. A large number of bitter compounds are known to be toxic. Thus, an ability to detect bitter compounds at low concentrations provided a certain evolutionary advantages. Even so, a number of non-toxic food and beverage products contain bitter compounds, such as coffee, tea, green leafy vegetables, and Citrus fruits. For many people, the concentration of bitter compounds in such foods and beverages falls below the level that induces an unpleasant reaction. But others may have a lower threshold for perceiving bitterness, and may experience reduced enjoyment when consuming these products. Adding sweeteners, such as sucrose, to such products can help to offset the bitterness. But adding sweeteners can increase the caloric content of the product, and may alter the taste too much for others who can easily withstand the mild natural bitterness of these products without sweetening.

Humans perceive bitterness when bitter compounds activate one or more of 25 different bitter taste receptors, which are generally located in the tongue. These bitter taste receptors are members of the TAS2R (taste receptor, type 2) family of receptors. Each of the 25 different bitter taste receptors contains a protein, whose sequence varies from one bitter taste receptor to another. These 25 different bitter taste receptors are generally referred to by number, such as TAS2R1, TAS2R3, TAS2R4, and the like. Different bitter compounds may activate different groups of bitter taste receptors. Thus, compounds that block the bitterness of certain bitter compounds may not work effectively at blocking the bitterness of others.

Citrus fruits contain a variety of compounds that humans generally perceive as bitter. Many of these compounds are present in high quantities in the peel or rind of Citrus fruits, but they can be present in the meat and pulp of the fruit as well. Some bitter receptor blockers work effectively at blocking the bitterness of certain foods or beverages, such as the bitter compounds present in coffee. But these compounds are not as effective at blocking the bitterness of Citrus products. Thus, there is a continuing need to discover compounds that can block the bitter taste receptors responsible for the tendency of humans to perceive certain Citrus products as bitter.

### SUMMARY

The present disclosure relates to the discovery of a collection of TAS2R taste receptors that are primarily responsible for the human perception of bitterness of certain compounds naturally present in Citrus.

In a first aspect, the disclosure provides a polypeptide sequence of SEQ ID NO: 1, or a functional fragment thereof, or a polypeptide sequence whose sequence is at least 90% equivalent, or at least 95% equivalent, or at least 97% equivalent, to either of the foregoing.

In a second aspect, the disclosure provides a polypeptide sequence of SEQ ID NO: 2, or a functional fragment thereof, or a polypeptide sequence whose sequence is at least 90% equivalent, or at least 95% equivalent, or at least 97% equivalent, to either of the foregoing.

In a third aspect, the disclosure provides a polypeptide sequence of SEQ ID NO: 3, or a functional fragment thereof, or a polypeptide sequence whose sequence is at least 90% equivalent, or at least 95% equivalent, or at least 97% equivalent, to either of the foregoing.

In a fourth aspect, the disclosure provides a polypeptide sequence of SEQ ID NO: 4, or a functional fragment thereof, or a polypeptide sequence whose sequence is at least 90% equivalent, or at least 95% equivalent, or at least 97% equivalent, to either of the foregoing.

In a fifth aspect, the disclosure provides a polypeptide sequence of SEQ ID NO: 5, or a functional fragment thereof, or a polypeptide sequence whose sequence is at least 90% equivalent, or at least 95% equivalent, or at least 97% equivalent, to either of the foregoing.

In a sixth aspect, the disclosure provides methods of identifying compounds that reduce bitter taste, the method comprising: introducing a test compound and a bitter compound to one or more taste receptor proteins, wherein the one or more taste receptor proteins are polypeptides that comprise: a polypeptide sequence of SEQ ID NO: 1, or a functional fragment thereof; a polypeptide sequence of SEQ ID NO: 2, or a functional fragment thereof; a polypeptide sequence of SEQ ID NO: 3, or a functional fragment thereof; a polypeptide sequence of SEQ ID NO: 4, or a functional fragment thereof; a polypeptide sequence of SEQ ID NO: 5, or a functional fragment thereof; or a polypeptide sequence whose sequence is at least 90% equivalent to any of the foregoing; and measuring a response of each of the one or more taste receptor proteins to the test compound by comparing an activity of the one or more taste receptor proteins to the bitter compound in the presence and the absence of the test compound. In some embodiments, the methods further comprise: identifying an active test compound that reduces bitter taste based on the measured response; and selecting the active test compound as a compound that reduces bitter taste.

In a seventh aspect, the disclosure provides uses of any identified or selected active test compounds of certain embodiments of the sixth aspect to reduce the bitterness of an ingestible composition. In some embodiments thereof, the ingestible composition is a composition derived from a Citrus fruit. In some embodiments, the ingestible composition is a naturally occurring composition. In some other embodiments, the ingestible composition is a non-naturally occurring composition. In some embodiments, the identified or selected active compounds are polymethoxyflavones (PMFs), such as PMFs derived from Citrus fruits. In some such embodiments, the PMFs are selected from the group consisting of sinensetin, 4′-hydroxy-sinensetin, quercetogetin, nobiletin, tangeretin, heptamethoxyflavone, and any combinations thereof.

In an eighth aspect, the disclosure provides uses of polymethoxyflavones (PMFs), such as PMFs derived from Citrus fruits, to reduce the bitterness of one or more bitter compounds. In some embodiments thereof, the one or more bitter compounds are comprised by an ingestible composition. In some embodiments thereof, the ingestible composition is a composition derived from Citrus fruit. In some embodiments, the ingestible composition is a naturally occurring composition. In some other embodiments, the ingestible composition is a non-naturally occurring composition. In some such embodiments, the PMFs are selected from the group consisting of sinensetin, 4′-hydroxy-sinensetin, quercetogetin, nobiletin, tangeretin, heptamethoxyflavone, and any combinations thereof. In some embodiments, the bitter compounds are compounds that modulates (e.g., activates) a polypeptide sequence of SEQ ID NO: 2, or a functional fragment thereof, or a polypeptide sequence whose sequence is at least 90% equivalent, or at least 95% equivalent, or at least 97% equivalent, to either of the foregoing. In some embodiments, the bitter compounds are limonoids, such as limonin, nomilin, nomilinic acid, or any combination thereof.

In a ninth aspect, the disclosure provides methods of reducing the bitterness of an ingestible composition, comprising introducing an amount (such as a bitterness-reducing effective amount) of any identified or selected active test compounds of certain embodiments of the sixth aspect to an ingestible composition. In some embodiments thereof, the ingestible composition comprises one or more compounds derived from a Citrus fruit. In some embodiments, the ingestible composition is a non-naturally occurring composition. In some embodiments, the identified or selected active compounds are polymethoxyflavones (PMFs), such as PMFs derived from Citrus fruits. In some such embodiments, the PMFs are selected from the group consisting of sinensetin,

4′-hydroxy-sinensetin, quercetogetin, nobiletin, tangeretin, heptamethoxyflavone, and any combinations thereof.

In a tenth aspect, the disclosure provides methods of reducing the bitterness of an ingestible composition, comprising introducing an amount (such as a bitterness-reducing effective amount) of polymethoxyflavones (PMFs) to an ingestible composition. In some embodiments thereof, the ingestible composition comprises one or more bitter compounds.

In some embodiments thereof, the ingestible composition is a composition derived from Citrus fruit. In some embodiments, the ingestible composition is a naturally occurring composition. In some other embodiments, the ingestible composition is a non-naturally occurring composition. In some such embodiments, the PMFs are selected from the group consisting of sinensetin, 4′-hydroxy-sinensetin, quercetogetin, nobiletin, tangeretin, heptamethoxyflavone, and any combinations thereof. In some embodiments, the bitter compounds are compounds that modulates (e.g., activates) a polypeptide sequence of SEQ ID NO: 2, or a functional fragment thereof, or a polypeptide sequence whose sequence is at least 90% equivalent, or at least 95% equivalent, or at least 97% equivalent, to either of the foregoing. In some embodiments, the bitter compounds are limonoids, such as limonin, nomilin, nomilinic acid, or any combination thereof.

In an eleventh aspect, the disclosure provides ingestible compositions comprising any identified or selected active test compounds of certain embodiments of the sixth aspect. In some embodiments thereof, the ingestible composition comprises one or more compounds derived from a Citrus fruit. In some embodiments, the ingestible composition is a non-naturally occurring composition.

In a twelfth aspect, the disclosure provides ingestible compositions comprising one or more polymethoxyflavones (PMFs). In some embodiments, the one or more PMFs are present in the ingestible composition in a bitterness-reducing effective amount. In some embodiments thereof, the ingestible composition comprises one or more bitter compounds. In some embodiments thereof, the ingestible composition is a composition derived from Citrus fruit. In some embodiments, the ingestible composition is a naturally occurring composition. In some other embodiments, the ingestible composition is a non-naturally occurring composition. In some such embodiments, the PMFs are selected from the group consisting of sinensetin, 4′-hydroxy-sinensetin, quercetogetin, nobiletin, tangeretin, heptamethoxyflavone, and any combinations thereof. In some embodiments, the bitter compounds are compounds that modulates (e.g., activates) a polypeptide sequence of SEQ ID NO: 2, or a functional fragment thereof, or a polypeptide sequence whose sequence is at least 90% equivalent, or at least 95% equivalent, or at least 97% equivalent, to either of the foregoing. In some embodiments, the bitter compounds are limonoids, such as limonin, nomilin, nomilinic acid, or any combination thereof.

In a thirteenth aspect, the disclosure provides flavored products comprising an ingestible composition of the eleventh aspect. In some embodiments, the flavored products are beverage products, such as soda, flavored water, tea, and the like. In some other embodiments, the flavored products are food products.

In a fourteenth aspect, the disclosure provides flavored products comprising an ingestible composition of the twelfth aspect. In some embodiments, the flavored products are beverage products, such as soda, flavored water, tea, and the like. In some other embodiments, the flavored products are food products.

In a fifteenth aspect, the disclosure provides methods for reducing the bitterness of Citrus compositions derived from Citrus fruit having Citrus greening disease, the method comprising: (a) deriving a Citrus composition from Citrus fruit, wherein at least a portion of the Citrus fruit has citrus greening disease; and (b) introducing to the Citrus composition a bitterness-reducing composition, which comprises polymethoxyflavones (PMFs), such as an amount (such as a bitterness-reducing effective amount) of polymethoxyflavones (PMFs). In some embodiments, the Citrus composition comprises an abnormally elevated concentration of one or more bitter compounds. In some such embodiments, the PMFs are selected from the group consisting of sinensetin, 4′-hydroxy-sinensetin, quercetogetin, nobiletin, tangeretin, heptamethoxyflavone, and any combinations thereof. In some embodiments, the bitter compounds are compounds that modulates (e.g., activates) a polypeptide sequence of SEQ ID NO: 2, or a functional fragment thereof, or a polypeptide sequence whose sequence is at least 90% equivalent, or at least 95% equivalent, or at least 97% equivalent, to either of the foregoing. In some embodiments, the bitter compounds are limonoids, such as limonin, nomilin, nomilinic acid, or any combination thereof. In some embodiments, the bitterness-reducing composition is derived from Citrus waste.

Further aspects, and embodiments thereof, are set forth below in the Detailed Description, the Drawings, the Abstract, and the Claims.

### BRIEF DESCRIPTION OF DRAWINGS

The following drawings are provided for purposes of illustrating various embodiments of the compositions and methods disclosed herein. The drawings are provided for illustrative purposes only, and are not intended to describe any preferred compositions or preferred methods, or to serve as a source of any limitations on the scope of the claimed inventions.

FIG. 1 shows six polymethoxyflavones (PMFs) that were discovered to have activity in antagonizing the activity of bitter compounds in Citrus.

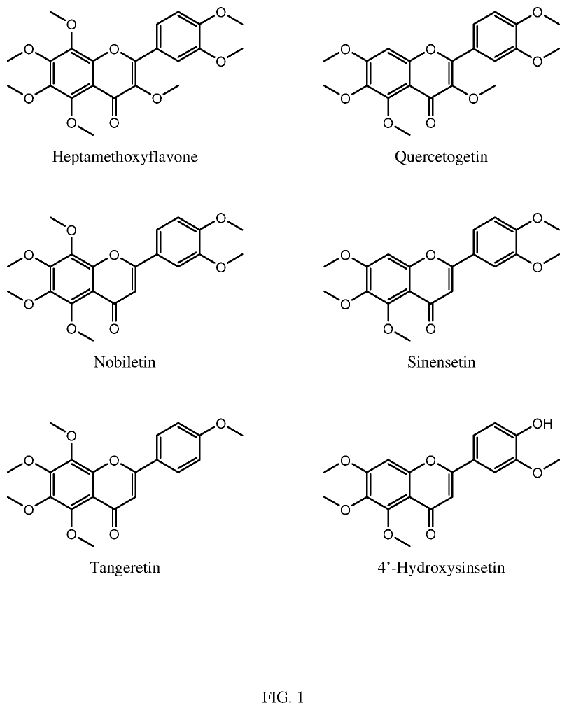

### DETAILED DESCRIPTION

The following Detailed Description sets forth various aspects and embodiments provided herein. The description is to be read from the perspective of the person of ordinary skill in the relevant art. Therefore, information that is well known to such ordinarily skilled artisans is not necessarily included.

### Definitions

The following terms and phrases have the meanings indicated below, unless otherwise provided herein. This disclosure may employ other terms and phrases not expressly defined herein. Such other terms and phrases have the meanings that they would possess within the context of this disclosure to those of ordinary skill in the art. In some instances, a term or phrase may be defined in the singular or plural. In such instances, it is understood that any term in the singular may include its plural counterpart and vice versa, unless expressly indicated to the contrary.

A “bitter compound” refers to a compound that elicits a detectable bitter flavor in a subject, e.g., a compound that activates a TAS2R receptor in vitro. A “bitter receptor blocker” or “bitter blocker” refers to a compound that antagonizes the activation of one or more TAS2R receptors in vitro by a bitter compound.

A “polymethoxyflavone” refers to flavone derivatives having three or more, or four or more, or five or more, methoxy substituents at the 3, 5, 6, 7, 8, 2′, 3′, 4′, 5′, and 6′ positions, where the numberings follow the usual flavone numberings, as shown below:

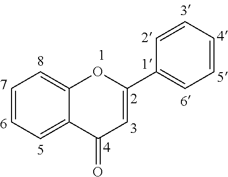 [SMILES: O=c1cc(-c2ccccc2)oc2ccccc12]

Non-limiting examples include the following compounds, with the left column giving the common name and the right column giving an alternate name.

| | |
|---|---|
| Sinensetin (SIN) | 5,6,7,3′,4′-Pentamethoxyflavone |
| Quercetogetin (HMQ) | 3,5,6,7,3′,4′-Hexamethoxyflavone |
| Nobiletin (NOB) | 5,6,7,8,3′,4′-Hexamethoxyflavone |
| Scutellarein | 5,6,7,4′-Tetramethoxyflavone |
| Tetramethylether (TMS) | |
| Heptamethoxyflavone (HMF) | 3,5,6,7,8,3′,4′-Heptamethoxyflavone |
| Tangeretin (TAN) | 5,6,7,8,4′-Pentamethoxyflavone |
| 5-OH SIN | 5-Hydroxy-6,7,3′,4′-tetramethoxyflavone |
| 5-OH HMQ | 5-Hydroxy-3,6,7,3′,4′- |
| | pentamethoxyflavone |
| 5-OH NOB | 5-Hydroxy-6,7,8,3′,4′- |
| | pentamethoxyflavone |
| 5-OH TMS | 5-Hydroxy-6,7,4′-trimethoxyflavone |
| 5-OH HMF | 5-Hydroxy-3,6,7,8,3′,4′- |
| | hexamethoxyflavone |
| 5-OH TAN | 5-Hydroxy-6,7,8,4′-tetramethoxyflavone |
| 4′-OH SIN | 4′-Hydroxy-5,6,7,3′-tetramethoxyflavone |
| 4′-OH HMF | 4′-Hydroxy-3,5,6,7,8,3′- |
| | hexamethoxyflavone |

A “functional fragment” refers to a portion of a polypeptide sequence to which the bitter compound binds. Polypeptide sequences often contain certain amino acids that do not actively participate in binding, but which may serve other purposes. In some instances, these non-functioning parts of the polypeptide sequence can be removed or partially replaced, while leaving the functional portion of the sequence intact. These modified proteins are said to comprise a functional fragment of the original polypeptide sequence.

As used herein, the singular forms “a,” “an,” and “the” include plural referents unless the context clearly dictates otherwise. For example, reference to “a substituent” encompasses a single substituent as well as two or more substituents, and the like.

As used herein, “for example,” “for instance,” “such as,” or “including” are meant to introduce examples that further clarify more general subject matter. Unless otherwise expressly indicated, such examples are provided only as an aid for understanding embodiments illustrated in the present disclosure, and are not meant to be limiting in any fashion. Nor do these phrases indicate any kind of preference for the disclosed embodiment.

As used herein, “comprise” or “comprises” or “comprising” or “comprised of” refer to groups that are open, meaning that the group can include additional members in addition to those expressly recited. For example, the phrase, “comprises A” means that A must be present, but that other members can be present too. The terms “include,” “have,” and “composed of” and their grammatical variants have the same meaning. In contrast, “consist of” or “consists of” or “consisting of” refer to groups that are closed. For example, the phrase “consists of A” means that A and only A is present.

As used herein, “optionally” means that the subsequently described event(s) may or may not occur. In some embodiments, the optional event does not occur. In some other embodiments, the optional event does occur one or more times.

As used herein, “or” is to be given its broadest reasonable interpretation, and is not to be limited to an either/or construction. Thus, the phrase “comprising A or B” means that A can be present and not B, or that B is present and not A, or that A and B are both present. Further, if A, for example, defines a class that can have multiple members, e.g., A₁ and A₂, then one or more members of the class can be present concurrently.

Other terms are defined in other portions of this description, even though not included in this subsection.

### Polypeptide Sequences Activated by Bitter Compounds

The present disclosure provides certain polypeptide sequences that are useful in the simulating in vitro the response that certain human bitter taste receptors would exhibit towards such compounds when ingested orally.

In a first aspect, the disclosure provides a polypeptide sequence of SEQ ID NO: 1, or a functional fragment thereof, or a polypeptide sequence whose sequence is at least 90% equivalent, or at least 95% equivalent, or at least 97% equivalent, to either of the foregoing. The polypeptide sequence corresponding to SEQ ID NO: 1 is set forth among the sequence listings filed herewith and incorporated by reference. For purposes of clarity and to avoid any confusion, the polypeptide sequence corresponding to SEQ ID NO: 1, starting with its N-terminus, is: MFSPADNIFIILITGEFILGILGNGYIALVNWIDWIKKKKISTVDYILT NLVIARICLISVMVVNGIVIVLNPDVYTKNKQQIVIFTFWTFANYLNMWITTCLN VFYFLKIASSSHPLFLWLKWKIDMVVHWILLGCFAISLLVSLIAAIVLSCDYRFHA IAKHKRNITEMFHVSKIPYFEPLTLFNLFAIVPFIVSLISFFLLVRSLWRHTKQIKLY ATGSRDPSTEVHVRAIKTMTSFIFFFFLYYISSILMTFSYLMTKYKLAVEFGEIA AILYPLGHSLILIVLNNKLRQTFVRMLTCRKIACMI, using the standard single-letter amino acid codes.

In a second aspect, the disclosure provides a polypeptide sequence of SEQ ID NO: 2, or a functional fragment thereof, or a polypeptide sequence whose sequence is at least 90% equivalent, or at least 95% equivalent, or at least 97% equivalent, to either of the foregoing. The polypeptide sequence corresponding to SEQ ID NO: 2 is set forth among the sequence listings filed herewith and incorporated by reference. For purposes of clarity and to avoid any confusion, the polypeptide sequence corresponding to SEQ ID NO: 2, starting with its N-terminus, is: MITFLPIIFSILIVVIFVIGNFANGFIALVNSIEWVKRQKISFVDQILT ALAVSRVGLLWVLLLHWYATQLNPAFYSVEVRITAYNVWAVTNHFSSWLATSL SMFYLLRIANFSNLIFLRIKRRVKSVVLVILLGPLLFLVCHLFVINMDETVWTKEYEG NVTWKIKLRSAMYHSNMTLTMLANFVPLTLTLISFLLLICSLCKHLKKMQLHGKGSQ DPSTKVHIKALQTVTSFLLLCAIYFLSMIISVCNLGRLEKQPVFMFCQAIIFSYPSTHPFI LILGNKKLKQIFLSVLRHVRYWVKDRSLRLHRFTRGALCVF, using the standard single-letter amino acid codes.

In a third aspect, the disclosure provides a polypeptide sequence of SEQ ID NO: 3, or a functional fragment thereof, or a polypeptide sequence whose sequence is at least 90% equivalent, or at least 95% equivalent, or at least 97% equivalent, to either of the foregoing. The polypeptide sequence corresponding to SEQ ID NO: 3 is set forth among the sequence listings filed herewith and incorporated by reference. For purposes of clarity and to avoid any confusion, the polypeptide sequence corresponding to SEQ ID NO: 3, starting with its N-terminus, is: MLTLTRIRTVSYEVRSTFLFISVLEFAVGFLTNAFVFLVNFWDVVK RQPLSNSDCVLLCLSISRLFLHGLLFLSAIQLTHFQKLSEPLNHSYQAIIMLWMIAN QANLWLAACLSLLYCSKLIRFSHTFLICLASWVSRKISQMLLGIILCSCICTVLCVW CFFSRPHFTVTTVLFMNNNTRLNWQIKDLNLFYSFLFCYLWSVPPFLLFLVSSGMLTV SLGRHMRTMKVYTRNSRDPSLEAHIKALKSLVSFFCFFVISSCAAFISVPLLILWRDG VMVCVGIMAACPSGHAAVLISGNAKLRRAVMTILLWAQSSLKVRADHKADSRTLC, using the standard single-letter amino acid codes.

In a fourth aspect, the disclosure provides a polypeptide sequence of SEQ ID NO: 4, or a functional fragment thereof, or a polypeptide sequence whose sequence is at least 90% equivalent, or at least 95% equivalent, or at least 97% equivalent, to either of the foregoing. The polypeptide sequence corresponding to SEQ ID NO: 4 is set forth among the sequence listings filed herewith and incorporated by reference. For purposes of clarity and to avoid any confusion, the polypeptide sequence corresponding to SEQ ID NO: 4, starting with its N-terminus, is: MMCFLLIISSILVVFAFVLGNVANGFIALVNIIDWVNTRKISSAEQILT ALVVSRIGLLWVMLFLWYATVFNSALYGLEVRIVASNAWAVTNHFSMWLAASLS IFCLLKIANFSNLISLHLKKRIKSVVLVILLGPLVFLICNLAVITMDERVWTKEYEGNV TWKIKLRNAIHLSSLTVTTLANLIPFTLSLICFLLLICSLCKHLKKMRLHSKGSQDPSTK VHIKALQTVTSFLMLFAIYFLCIITSTWNLRTQQSKLVLLLCQTVAIMYPSFHSFILIMG SRKLKQTFLSVLWQMTR, using the standard single-letter amino acid codes.

In a fifth aspect, the disclosure provides a polypeptide sequence of SEQ ID NO: 5, or a functional fragment thereof, or a polypeptide sequence whose sequence is at least 90% equivalent, or at least 95% equivalent, or at least 97% equivalent, to either of the foregoing. The polypeptide sequence corresponding to SEQ ID NO: 5 is set forth among the sequence listings filed herewith and incorporated by reference. For purposes of clarity and to avoid any confusion, the polypeptide sequence corresponding to SEQ ID NO: 5, starting with its N-terminus, is: MITFLPIIFSILIVVTFVIGNFANGFIALVNSIEWFKRQKISFADQILTAL AVSRVGLLWVLVLNWYATELNPAFNSIEVRITAYNVWAVINHFSNWLATSLSIFY LLKIANFSNLIFLHLKRRVKSVVLVILLGPLLFLVCHLFVINMNQIIWTKEYEGNMTW KIKLRSAMYLSNTTVTILANLVPFTLTLISFLLLICSLCKHLKKMQLHGKGSQDPSMK VHIKALQTVTSFLLLCAIYFLSIIMSVWSFESLENKPVFMFCEAIAFSYPSTHPFILIWG NKKLKQTFLSVLWHVRYWVKGEKPSSS, using the standard single-letter amino acid codes.

The polypeptide sequences of the foregoing aspects and embodiments can be present in any suitable composition. In some embodiments, one or more polypeptide sequences of the foregoing aspects and embodiments is present in a non-naturally occurring composition, such as an in vitro assay. In some further such embodiments, the polypeptide sequences of the foregoing aspects and embodiments are expressed on the surface of cells, such as on the cells of a eukaryotic cell line.

### Screening Methods

In a sixth aspect, the disclosure provides methods of identifying compounds that reduce bitter taste, the method comprising: introducing a test compound and a bitter compound to one or more taste receptor proteins, wherein the one or more taste receptor proteins are polypeptides that comprise: a polypeptide sequence of SEQ ID NO: 1, or a functional fragment thereof; a polypeptide sequence of SEQ ID NO: 2, or a functional fragment thereof; a polypeptide sequence of SEQ ID NO: 3, or a functional fragment thereof; a polypeptide sequence of SEQ ID NO: 4, or a functional fragment thereof; a polypeptide sequence of SEQ ID NO: 5, or a functional fragment thereof; or a polypeptide sequence whose sequence is at least 90% equivalent to any of the foregoing; and measuring a response of each of the one or more taste receptor proteins to the test compound by comparing an activity of the one or more taste receptor proteins to the bitter compound in the presence and the absence of the test compound.

In some embodiments thereof, the introducing step comprises introducing a test compound and a bitter compound to a taste receptor protein, wherein the taste receptor protein is a polypeptide that comprises: a polypeptide sequence of SEQ ID NO: 1, or a functional fragment thereof, or a polypeptide sequence whose sequence is at least 90% equivalent, or at least 95% equivalent, or at least 97% equivalent, to either of the foregoing. In some further embodiments, the taste receptor protein is a polypeptide that comprises: a polypeptide sequence of SEQ ID NO: 1, or a polypeptide sequence whose sequence is at least 90% equivalent, or at least 95% equivalent, or at least 97% equivalent, thereto. In some embodiments, the polypeptide sequence of SEQ ID NO: 1 can be referred to as hT2R80.

In some further embodiments of any of the foregoing embodiments, the introducing step further comprises introducing a test compound and a bitter compound to a taste receptor protein, wherein the taste receptor protein is a polypeptide that comprises: a polypeptide sequence of SEQ ID NO: 2, or a functional fragment thereof, or a polypeptide sequence whose sequence is at least 90% equivalent, or at least 95% equivalent, or at least 97% equivalent, to either of the foregoing. In some further embodiments, the taste receptor protein is a polypeptide that comprises: a polypeptide sequence of SEQ ID NO: 2, or a polypeptide sequence whose sequence is at least 90% equivalent, or at least 95% equivalent, or at least 97% equivalent, thereto. In some embodiments, the polypeptide sequence of SEQ ID NO: 2 can be referred to as hT2R44.

In some further embodiments of any of the foregoing embodiments, the introducing step further comprises introducing a test compound and a bitter compound to a taste receptor protein, wherein the taste receptor protein is a polypeptide that comprises: a polypeptide sequence of SEQ ID NO: 3, or a functional fragment thereof, or a polypeptide sequence whose sequence is at least 90% equivalent, or at least 95% equivalent, or at least 97% equivalent, to either of the foregoing. In some further embodiments, the taste receptor protein is a polypeptide that comprises: a polypeptide sequence of SEQ ID NO: 3, or a polypeptide sequence whose sequence is at least 90% equivalent, or at least 95% equivalent, or at least 97% equivalent, thereto. In some embodiments, the polypeptide sequence of SEQ ID NO: 3 can be referred to as hT2R51.

In some further embodiments of any of the foregoing embodiments, the introducing step further comprises introducing a test compound and a bitter compound to a taste receptor protein, wherein the taste receptor protein is a polypeptide that comprises: a polypeptide sequence of SEQ ID NO: 4, or a functional fragment thereof, or a polypeptide sequence whose sequence is at least 90% equivalent, or at least 95% equivalent, or at least 97% equivalent, to either of the foregoing. In some further embodiments, the taste receptor protein is a polypeptide that comprises: a polypeptide sequence of SEQ ID NO: 4, or a polypeptide sequence whose sequence is at least 90% equivalent, or at least 95% equivalent, or at least 97% equivalent, thereto. In some embodiments, the polypeptide sequence of SEQ ID NO: 4 can be referred to as hT2R65.

In some further embodiments of any of the foregoing embodiments, the introducing step further comprises introducing a test compound and a bitter compound to a taste receptor protein, wherein the taste receptor protein is a polypeptide that comprises: a polypeptide sequence of SEQ ID NO: 5, or a functional fragment thereof, or a polypeptide sequence whose sequence is at least 90% equivalent, or at least 95% equivalent, or at least 97% equivalent, to either of the foregoing. In some further embodiments, the taste receptor protein is a polypeptide that comprises: a polypeptide sequence of SEQ ID NO: 5, or a polypeptide sequence whose sequence is at least 90% equivalent, or at least 95% equivalent, or at least 97% equivalent, thereto. In some embodiments, the polypeptide sequence of SEQ ID NO: 5 can be referred to as hT2R75.

Note that the introducing step can include introducing the test compound and a bitter compound to any combination of the peptide sequences of the five foregoing paragraphs. This can be carried out in any suitable way. For example, in some non-limiting instances, when introducing the test compound and the bitter compound to two or more different taste receptor proteins, the introducing can be carried out in a single cell-based assay. But in some other non-limiting instances, when introducing the test compound and the bitter compound to two or more different taste receptor proteins, the introducing can be carried out in a two or more cell-based assays, such as one separate assay for each taste receptor protein.

The present disclosure contemplates screening the test compound against any combination of two or more, or three or more, or four or more, or five different taste receptor proteins associated with each of the five different polypeptide sequences set forth herein.

Thus, in some embodiments, the introducing comprises introducing a test compound and a bitter-taste compound to each of a first taste receptor protein and a second taste receptor protein (for example, either in the same assay or different assays); wherein the first taste receptor protein is a polypeptide that comprises a polypeptide sequence of SEQ ID NO: 1, a functional fragment thereof, or a polypeptide whose sequence is at least 90% equivalent, or at least 95% equivalent, or at least 97% equivalent, to either of the foregoing; wherein the second taste receptor protein is a polypeptide that comprises a polypeptide sequence of SEQ ID NO: 2, a functional fragment thereof, or a polypeptide whose sequence is at least 90% equivalent, or at least 95% equivalent, or at least 97% equivalent, to either of the foregoing; and wherein the measuring comprises measuring a response of each of the first receptor protein and the second receptor protein to the test compound by comparing an activity of each of the first receptor protein and the second receptor protein to the bitter compound in the presence and the absence of the test compound. In some further embodiments, the first taste receptor protein is a polypeptide that comprises a polypeptide sequence of SEQ ID NO: 1, or a polypeptide whose sequence is at least 90% equivalent, or at least 95% equivalent, or at least 97% equivalent, thereto, and the second taste receptor protein is a polypeptide that comprises a polypeptide sequence of SEQ ID NO: 2, or a polypeptide whose sequence is at least 90% equivalent, or at least 95% equivalent, or at least 97% equivalent, thereto.

Thus, in some embodiments, the introducing comprises introducing a test compound and a bitter-taste compound to each of a first taste receptor protein and a second taste receptor protein (for example, either in the same assay or different assays); wherein the first taste receptor protein is a polypeptide that comprises a polypeptide sequence of SEQ ID NO: 1, a functional fragment thereof, or a polypeptide whose sequence is at least 90% equivalent, or at least 95% equivalent, or at least 97% equivalent, to either of the foregoing; wherein the second taste receptor protein is a polypeptide that comprises a polypeptide sequence of SEQ ID NO: 3, a functional fragment thereof, or a polypeptide whose sequence is at least 90% equivalent, or at least 95% equivalent, or at least 97% equivalent, to either of the foregoing; and wherein the measuring comprises measuring a response of each of the first receptor protein and the second receptor protein to the test compound by comparing an activity of each of the first receptor protein and the second receptor protein to the bitter compound in the presence and the absence of the test compound. In some further embodiments, the first taste receptor protein is a polypeptide that comprises a polypeptide sequence of SEQ ID NO: 1, or a polypeptide whose sequence is at least 90% equivalent, or at least 95% equivalent, or at least 97% equivalent, thereto, and the second taste receptor protein is a polypeptide that comprises a polypeptide sequence of SEQ ID NO: 3, or a polypeptide whose sequence is at least 90% equivalent, or at least 95% equivalent, or at least 97% equivalent, thereto.

Thus, in some embodiments, the introducing comprises introducing a test compound and a bitter-taste compound to each of a first taste receptor protein and a second taste receptor protein (for example, either in the same assay or different assays); wherein the first taste receptor protein is a polypeptide that comprises a polypeptide sequence of SEQ ID NO: 1, a functional fragment thereof, or a polypeptide whose sequence is at least 90% equivalent, or at least 95% equivalent, or at least 97% equivalent, to either of the foregoing; wherein the second taste receptor protein is a polypeptide that comprises a polypeptide sequence of SEQ ID NO: 4, a functional fragment thereof, or a polypeptide whose sequence is at least 90% equivalent, or at least 95% equivalent, or at least 97% equivalent, to either of the foregoing; and wherein the measuring comprises measuring a response of each of the first receptor protein and the second receptor protein to the test compound by comparing an activity of each of the first receptor protein and the second receptor protein to the bitter compound in the presence and the absence of the test compound. In some further embodiments, the first taste receptor protein is a polypeptide that comprises a polypeptide sequence of SEQ ID NO: 1, or a polypeptide whose sequence is at least 90% equivalent, or at least 95% equivalent, or at least 97% equivalent, thereto, and the second taste receptor protein is a polypeptide that comprises a polypeptide sequence of SEQ ID NO: 4, or a polypeptide whose sequence is at least 90% equivalent, or at least 95% equivalent, or at least 97% equivalent, thereto.

Thus, in some embodiments, the introducing comprises introducing a test compound and a bitter-taste compound to each of a first taste receptor protein and a second taste receptor protein (for example, either in the same assay or different assays); wherein the first taste receptor protein is a polypeptide that comprises a polypeptide sequence of SEQ ID NO: 1, a functional fragment thereof, or a polypeptide whose sequence is at least 90% equivalent, or at least 95% equivalent, or at least 97% equivalent, to either of the foregoing; wherein the second taste receptor protein is a polypeptide that comprises a polypeptide sequence of SEQ ID NO: 5, a functional fragment thereof, or a polypeptide whose sequence is at least 90% equivalent, or at least 95% equivalent, or at least 97% equivalent, to either of the foregoing; and wherein the measuring comprises measuring a response of each of the first receptor protein and the second receptor protein to the test compound by comparing an activity of each of the first receptor protein and the second receptor protein to the bitter compound in the presence and the absence of the test compound. In some further embodiments, the first taste receptor protein is a polypeptide that comprises a polypeptide sequence of SEQ ID NO: 1, or a polypeptide whose sequence is at least 90% equivalent, or at least 95% equivalent, or at least 97% equivalent, thereto, and the second taste receptor protein is a polypeptide that comprises a polypeptide sequence of SEQ ID NO: 5, or a polypeptide whose sequence is at least 90% equivalent, or at least 95% equivalent, or at least 97% equivalent, thereto.

Thus, in some embodiments, the introducing comprises introducing a test compound and a bitter-taste compound to each of a first taste receptor protein and a second taste receptor protein (for example, either in the same assay or different assays); wherein the first taste receptor protein is a polypeptide that comprises a polypeptide sequence of SEQ ID NO: 2, a functional fragment thereof, or a polypeptide whose sequence is at least 90% equivalent, or at least 95% equivalent, or at least 97% equivalent, to either of the foregoing; wherein the second taste receptor protein is a polypeptide that comprises a polypeptide sequence of SEQ ID NO: 3, a functional fragment thereof, or a polypeptide whose sequence is at least 90% equivalent, or at least 95% equivalent, or at least 97% equivalent, to either of the foregoing; and wherein the measuring comprises measuring a response of each of the first receptor protein and the second receptor protein to the test compound by comparing an activity of each of the first receptor protein and the second receptor protein to the bitter compound in the presence and the absence of the test compound. In some further embodiments, the first taste receptor protein is a polypeptide that comprises a polypeptide sequence of SEQ ID NO: 2, or a polypeptide whose sequence is at least 90% equivalent, or at least 95% equivalent, or at least 97% equivalent, thereto, and the second taste receptor protein is a polypeptide that comprises a polypeptide sequence of SEQ ID NO: 3, or a polypeptide whose sequence is at least 90% equivalent, or at least 95% equivalent, or at least 97% equivalent, thereto.

Thus, in some embodiments, the introducing comprises introducing a test compound and a bitter-taste compound to each of a first taste receptor protein and a second taste receptor protein (for example, either in the same assay or different assays); wherein the first taste receptor protein is a polypeptide that comprises a polypeptide sequence of SEQ ID NO: 2, a functional fragment thereof, or a polypeptide whose sequence is at least 90% equivalent, or at least 95% equivalent, or at least 97% equivalent, to either of the foregoing; wherein the second taste receptor protein is a polypeptide that comprises a polypeptide sequence of SEQ ID NO: 4, a functional fragment thereof, or a polypeptide whose sequence is at least 90% equivalent, or at least 95% equivalent, or at least 97% equivalent, to either of the foregoing; and wherein the measuring comprises measuring a response of each of the first receptor protein and the second receptor protein to the test compound by comparing an activity of each of the first receptor protein and the second receptor protein to the bitter compound in the presence and the absence of the test compound. In some further embodiments, the first taste receptor protein is a polypeptide that comprises a polypeptide sequence of SEQ ID NO: 2, or a polypeptide whose sequence is at least 90% equivalent, or at least 95% equivalent, or at least 97% equivalent, thereto, and the second taste receptor protein is a polypeptide that comprises a polypeptide sequence of SEQ ID NO: 4, or a polypeptide whose sequence is at least 90% equivalent, or at least 95% equivalent, or at least 97% equivalent, thereto.

Thus, in some embodiments, the introducing comprises introducing a test compound and a bitter-taste compound to each of a first taste receptor protein and a second taste receptor protein (for example, either in the same assay or different assays); wherein the first taste receptor protein is a polypeptide that comprises a polypeptide sequence of SEQ ID NO: 2, a functional fragment thereof, or a polypeptide whose sequence is at least 90% equivalent, or at least 95% equivalent, or at least 97% equivalent, to either of the foregoing; wherein the second taste receptor protein is a polypeptide that comprises a polypeptide sequence of SEQ ID NO: 5, a functional fragment thereof, or a polypeptide whose sequence is at least 90% equivalent, or at least 95% equivalent, or at least 97% equivalent, to either of the foregoing; and wherein the measuring comprises measuring a response of each of the first receptor protein and the second receptor protein to the test compound by comparing an activity of each of the first receptor protein and the second receptor protein to the bitter compound in the presence and the absence of the test compound. In some further embodiments, the first taste receptor protein is a polypeptide that comprises a polypeptide sequence of SEQ ID NO: 2, or a polypeptide whose sequence is at least 90% equivalent, or at least 95% equivalent, or at least 97% equivalent, thereto, and the second taste receptor protein is a polypeptide that comprises a polypeptide sequence of SEQ ID NO: 5, or a polypeptide whose sequence is at least 90% equivalent, or at least 95% equivalent, or at least 97% equivalent, thereto.

Thus, in some embodiments, the introducing comprises introducing a test compound and a bitter-taste compound to each of a first taste receptor protein and a second taste receptor protein (for example, either in the same assay or different assays); wherein the first taste receptor protein is a polypeptide that comprises a polypeptide sequence of SEQ ID NO: 3, a functional fragment thereof, or a polypeptide whose sequence is at least 90% equivalent, or at least 95% equivalent, or at least 97% equivalent, to either of the foregoing; wherein the second taste receptor protein is a polypeptide that comprises a polypeptide sequence of SEQ ID NO: 4, a functional fragment thereof, or a polypeptide whose sequence is at least 90% equivalent, or at least 95% equivalent, or at least 97% equivalent, to either of the foregoing; and wherein the measuring comprises measuring a response of each of the first receptor protein and the second receptor protein to the test compound by comparing an activity of each of the first receptor protein and the second receptor protein to the bitter compound in the presence and the absence of the test compound. In some further embodiments, the first taste receptor protein is a polypeptide that comprises a polypeptide sequence of SEQ ID NO: 3, or a polypeptide whose sequence is at least 90% equivalent, or at least 95% equivalent, or at least 97% equivalent, thereto, and the second taste receptor protein is a polypeptide that comprises a polypeptide sequence of SEQ ID NO: 4, or a polypeptide whose sequence is at least 90% equivalent, or at least 95% equivalent, or at least 97% equivalent, thereto.

Thus, in some embodiments, the introducing comprises introducing a test compound and a bitter-taste compound to each of a first taste receptor protein and a second taste receptor protein (for example, either in the same assay or different assays); wherein the first taste receptor protein is a polypeptide that comprises a polypeptide sequence of SEQ ID NO: 3, a functional fragment thereof, or a polypeptide whose sequence is at least 90% equivalent, or at least 95% equivalent, or at least 97% equivalent, to either of the foregoing; wherein the second taste receptor protein is a polypeptide that comprises a polypeptide sequence of SEQ ID NO: 5, a functional fragment thereof, or a polypeptide whose sequence is at least 90% equivalent, or at least 95% equivalent, or at least 97% equivalent, to either of the foregoing; and wherein the measuring comprises measuring a response of each of the first receptor protein and the second receptor protein to the test compound by comparing an activity of each of the first receptor protein and the second receptor protein to the bitter compound in the presence and the absence of the test compound. In some further embodiments, the first taste receptor protein is a polypeptide that comprises a polypeptide sequence of SEQ ID NO: 3, or a polypeptide whose sequence is at least 90% equivalent, or at least 95% equivalent, or at least 97% equivalent, thereto, and the second taste receptor protein is a polypeptide that comprises a polypeptide sequence of SEQ ID NO: 5, or a polypeptide whose sequence is at least 90% equivalent, or at least 95% equivalent, or at least 97% equivalent, thereto.

Thus, in some embodiments, the introducing comprises introducing a test compound and a bitter-taste compound to each of a first taste receptor protein and a second taste receptor protein (for example, either in the same assay or different assays); wherein the first taste receptor protein is a polypeptide that comprises a polypeptide sequence of SEQ ID NO: 4, a functional fragment thereof, or a polypeptide whose sequence is at least 90% equivalent, or at least 95% equivalent, or at least 97% equivalent, to either of the foregoing; wherein the second taste receptor protein is a polypeptide that comprises a polypeptide sequence of SEQ ID NO: 5, a functional fragment thereof, or a polypeptide whose sequence is at least 90% equivalent, or at least 95% equivalent, or at least 97% equivalent, to either of the foregoing; and wherein the measuring comprises measuring a response of each of the first receptor protein and the second receptor protein to the test compound by comparing an activity of each of the first receptor protein and the second receptor protein to the bitter compound in the presence and the absence of the test compound. In some further embodiments, the first taste receptor protein is a polypeptide that comprises a polypeptide sequence of SEQ ID NO: 4, or a polypeptide whose sequence is at least 90% equivalent, or at least 95% equivalent, or at least 97% equivalent, thereto, and the second taste receptor protein is a polypeptide that comprises a polypeptide sequence of SEQ ID NO: 5, or a polypeptide whose sequence is at least 90% equivalent, or at least 95% equivalent, or at least 97% equivalent, thereto.

In like manner, the introducing and measuring can involve any combination of three different taste receptor proteins, such as:

a polypeptide that comprises a polypeptide sequence of SEQ ID NO: 1, a functional fragment thereof, or a polypeptide whose sequence is at least 90% equivalent, or at least 95% equivalent, or at least 97% equivalent, to either of the foregoing; a polypeptide that comprises a polypeptide sequence of SEQ ID NO: 2, a functional fragment thereof, or a polypeptide whose sequence is at least 90% equivalent, or at least 95% equivalent, or at least 97% equivalent, to either of the foregoing; and a polypeptide that comprises a polypeptide sequence of SEQ ID NO: 3, a functional fragment thereof, or a polypeptide whose sequence is at least 90% equivalent, or at least 95% equivalent, or at least 97% equivalent, to either of the foregoing, with corresponding measuring steps for each of the three different polypeptide sequences; or

a polypeptide that comprises a polypeptide sequence of SEQ ID NO: 1, a functional fragment thereof, or a polypeptide whose sequence is at least 90% equivalent, or at least 95% equivalent, or at least 97% equivalent, to either of the foregoing; a polypeptide that comprises a polypeptide sequence of SEQ ID NO: 2, a functional fragment thereof, or a polypeptide whose sequence is at least 90% equivalent, or at least 95% equivalent, or at least 97% equivalent, to either of the foregoing; and a polypeptide that comprises a polypeptide sequence of SEQ ID NO: 4, a functional fragment thereof, or a polypeptide whose sequence is at least 90% equivalent, or at least 95% equivalent, or at least 97% equivalent, to either of the foregoing, with corresponding measuring steps for each of the three different polypeptide sequences; or

a polypeptide that comprises a polypeptide sequence of SEQ ID NO: 1, a functional fragment thereof, or a polypeptide whose sequence is at least 90% equivalent, or at least 95% equivalent, or at least 97% equivalent, to either of the foregoing; a polypeptide that comprises a polypeptide sequence of SEQ ID NO: 2, a functional fragment thereof, or a polypeptide whose sequence is at least 90% equivalent, or at least 95% equivalent, or at least 97% equivalent, to either of the foregoing; and a polypeptide that comprises a polypeptide sequence of SEQ ID NO: 5, a functional fragment thereof, or a polypeptide whose sequence is at least 90% equivalent, or at least 95% equivalent, or at least 97% equivalent, to either of the foregoing, with corresponding measuring steps for each of the three different polypeptide sequences; or

a polypeptide that comprises a polypeptide sequence of SEQ ID NO: 2, a functional fragment thereof, or a polypeptide whose sequence is at least 90% equivalent, or at least 95% equivalent, or at least 97% equivalent, to either of the foregoing; a polypeptide that comprises a polypeptide sequence of SEQ ID NO: 3, a functional fragment thereof, or a polypeptide whose sequence is at least 90% equivalent, or at least 95% equivalent, or at least 97% equivalent, to either of the foregoing; and a polypeptide that comprises a polypeptide sequence of SEQ ID NO: 4, a functional fragment thereof, or a polypeptide whose sequence is at least 90% equivalent, or at least 95% equivalent, or at least 97% equivalent, to either of the foregoing, with corresponding measuring steps for each of the three different polypeptide sequences; or

a polypeptide that comprises a polypeptide sequence of SEQ ID NO: 1, a functional fragment thereof, or a polypeptide whose sequence is at least 90% equivalent, or at least 95% equivalent, or at least 97% equivalent, to either of the foregoing; a polypeptide that comprises a polypeptide sequence of SEQ ID NO: 2, a functional fragment thereof, or a polypeptide whose sequence is at least 90% equivalent, or at least 95% equivalent, or at least 97% equivalent, to either of the foregoing; and a polypeptide that comprises a polypeptide sequence of SEQ ID NO: 5, a functional fragment thereof, or a polypeptide whose sequence is at least 90% equivalent, or at least 95% equivalent, or at least 97% equivalent, to either of the foregoing, with corresponding measuring steps for each of the three different polypeptide sequences; or

a polypeptide that comprises a polypeptide sequence of SEQ ID NO: 1, a functional fragment thereof, or a polypeptide whose sequence is at least 90% equivalent, or at least 95% equivalent, or at least 97% equivalent, to either of the foregoing; a polypeptide that comprises a polypeptide sequence of SEQ ID NO: 3, a functional fragment thereof, or a polypeptide whose sequence is at least 90% equivalent, or at least 95% equivalent, or at least 97% equivalent, to either of the foregoing; and a polypeptide that comprises a polypeptide sequence of SEQ ID NO: 5, a functional fragment thereof, or a polypeptide whose sequence is at least 90% equivalent, or at least 95% equivalent, or at least 97% equivalent, to either of the foregoing, with corresponding measuring steps for each of the three different polypeptide sequences; or

a polypeptide that comprises a polypeptide sequence of SEQ ID NO: 2, a functional fragment thereof, or a polypeptide whose sequence is at least 90% equivalent, or at least 95% equivalent, or at least 97% equivalent, to either of the foregoing; a polypeptide that comprises a polypeptide sequence of SEQ ID NO: 3, a functional fragment thereof, or a polypeptide whose sequence is at least 90% equivalent, or at least 95% equivalent, or at least 97% equivalent, to either of the foregoing; and a polypeptide that comprises a polypeptide sequence of SEQ ID NO: 5, a functional fragment thereof, or a polypeptide whose sequence is at least 90% equivalent, or at least 95% equivalent, or at least 97% equivalent, to either of the foregoing, with corresponding measuring steps for each of the three different polypeptide sequences; or

a polypeptide that comprises a polypeptide sequence of SEQ ID NO: 1, a functional fragment thereof, or a polypeptide whose sequence is at least 90% equivalent, or at least 95% equivalent, or at least 97% equivalent, to either of the foregoing; a polypeptide that comprises a polypeptide sequence of SEQ ID NO: 5, a functional fragment thereof, or a polypeptide whose sequence is at least 90% equivalent, or at least 95% equivalent, or at least 97% equivalent, to either of the foregoing; and a polypeptide that comprises a polypeptide sequence of SEQ ID NO: 5, a functional fragment thereof, or a polypeptide whose sequence is at least 90% equivalent, or at least 95% equivalent, or at least 97% equivalent, to either of the foregoing, with corresponding measuring steps for each of the three different polypeptide sequences; or

a polypeptide that comprises a polypeptide sequence of SEQ ID NO: 2, a functional fragment thereof, or a polypeptide whose sequence is at least 90% equivalent, or at least 95% equivalent, or at least 97% equivalent, to either of the foregoing; a polypeptide that comprises a polypeptide sequence of SEQ ID NO: 4, a functional fragment thereof, or a polypeptide whose sequence is at least 90% equivalent, or at least 95% equivalent, or at least 97% equivalent, to either of the foregoing; and a polypeptide that comprises a polypeptide sequence of SEQ ID NO: 5, a functional fragment thereof, or a polypeptide whose sequence is at least 90% equivalent, or at least 95% equivalent, or at least 97% equivalent, to either of the foregoing, with corresponding measuring steps for each of the three different polypeptide sequences; or

a polypeptide that comprises a polypeptide sequence of SEQ ID NO: 3, a functional fragment thereof, or a polypeptide whose sequence is at least 90% equivalent, or at least 95% equivalent, or at least 97% equivalent, to either of the foregoing; a polypeptide that comprises a polypeptide sequence of SEQ ID NO: 4, a functional fragment thereof, or a polypeptide whose sequence is at least 90% equivalent, or at least 95% equivalent, or at least 97% equivalent, to either of the foregoing; and a polypeptide that comprises a polypeptide sequence of SEQ ID NO: 5, a functional fragment thereof, or a polypeptide whose sequence is at least 90% equivalent, or at least 95% equivalent, or at least 97% equivalent, to either of the foregoing, with corresponding measuring steps for each of the three different polypeptide sequences.

In like manner, the introducing and measuring can involve any combination of four different taste receptor proteins, such as:

a polypeptide that comprises a polypeptide sequence of SEQ ID NO: 1, a functional fragment thereof, or a polypeptide whose sequence is at least 90% equivalent, or at least 95% equivalent, or at least 97% equivalent, to either of the foregoing; a polypeptide that comprises a polypeptide sequence of SEQ ID NO: 2, a functional fragment thereof, or a polypeptide whose sequence is at least 90% equivalent, or at least 95% equivalent, or at least 97% equivalent, to either of the foregoing; a polypeptide that comprises a polypeptide sequence of SEQ ID NO: 3, a functional fragment thereof, or a polypeptide whose sequence is at least 90% equivalent, or at least 95% equivalent, or at least 97% equivalent, to either of the foregoing, and a polypeptide that comprises a polypeptide sequence of SEQ ID NO: 4, a functional fragment thereof, or a polypeptide whose sequence is at least 90% equivalent, or at least 95% equivalent, or at least 97% equivalent, to either of the foregoing, with corresponding measuring steps for each of the three different polypeptide sequences; or

a polypeptide that comprises a polypeptide sequence of SEQ ID NO: 1, a functional fragment thereof, or a polypeptide whose sequence is at least 90% equivalent, or at least 95% equivalent, or at least 97% equivalent, to either of the foregoing; a polypeptide that comprises a polypeptide sequence of SEQ ID NO: 2, a functional fragment thereof, or a polypeptide whose sequence is at least 90% equivalent, or at least 95% equivalent, or at least 97% equivalent, to either of the foregoing; a polypeptide that comprises a polypeptide sequence of SEQ ID NO: 3, a functional fragment thereof, or a polypeptide whose sequence is at least 90% equivalent, or at least 95% equivalent, or at least 97% equivalent, to either of the foregoing, and a polypeptide that comprises a polypeptide sequence of SEQ ID NO: 5, a functional fragment thereof, or a polypeptide whose sequence is at least 90% equivalent, or at least 95% equivalent, or at least 97% equivalent, to either of the foregoing, with corresponding measuring steps for each of the three different polypeptide sequences; or

a polypeptide that comprises a polypeptide sequence of SEQ ID NO: 1, a functional fragment thereof, or a polypeptide whose sequence is at least 90% equivalent, or at least 95% equivalent, or at least 97% equivalent, to either of the foregoing; a polypeptide that comprises a polypeptide sequence of SEQ ID NO: 2, a functional fragment thereof, or a polypeptide whose sequence is at least 90% equivalent, or at least 95% equivalent, or at least 97% equivalent, to either of the foregoing; a polypeptide that comprises a polypeptide sequence of SEQ ID NO: 4, a functional fragment thereof, or a polypeptide whose sequence is at least 90% equivalent, or at least 95% equivalent, or at least 97% equivalent, to either of the foregoing, and a polypeptide that comprises a polypeptide sequence of SEQ ID NO: 5, a functional fragment thereof, or a polypeptide whose sequence is at least 90% equivalent, or at least 95% equivalent, or at least 97% equivalent, to either of the foregoing, with corresponding measuring steps for each of the three different polypeptide sequences; or

a polypeptide that comprises a polypeptide sequence of SEQ ID NO: 1, a functional fragment thereof, or a polypeptide whose sequence is at least 90% equivalent, or at least 95% equivalent, or at least 97% equivalent, to either of the foregoing; a polypeptide that comprises a polypeptide sequence of SEQ ID NO: 3, a functional fragment thereof, or a polypeptide whose sequence is at least 90% equivalent, or at least 95% equivalent, or at least 97% equivalent, to either of the foregoing; a polypeptide that comprises a polypeptide sequence of SEQ ID NO: 4, a functional fragment thereof, or a polypeptide whose sequence is at least 90% equivalent, or at least 95% equivalent, or at least 97% equivalent, to either of the foregoing, and a polypeptide that comprises a polypeptide sequence of SEQ ID NO: 5, a functional fragment thereof, or a polypeptide whose sequence is at least 90% equivalent, or at least 95% equivalent, or at least 97% equivalent, to either of the foregoing, with corresponding measuring steps for each of the three different polypeptide sequences; or

a polypeptide that comprises a polypeptide sequence of SEQ ID NO: 2, a functional fragment thereof, or a polypeptide whose sequence is at least 90% equivalent, or at least 95% equivalent, or at least 97% equivalent, to either of the foregoing; a polypeptide that comprises a polypeptide sequence of SEQ ID NO: 3, a functional fragment thereof, or a polypeptide whose sequence is at least 90% equivalent, or at least 95% equivalent, or at least 97% equivalent, to either of the foregoing; a polypeptide that comprises a polypeptide sequence of SEQ ID NO: 4, a functional fragment thereof, or a polypeptide whose sequence is at least 90% equivalent, or at least 95% equivalent, or at least 97% equivalent, to either of the foregoing, and a polypeptide that comprises a polypeptide sequence of SEQ ID NO: 5, a functional fragment thereof, or a polypeptide whose sequence is at least 90% equivalent, or at least 95% equivalent, or at least 97% equivalent, to either of the foregoing, with corresponding measuring steps for each of the three different polypeptide sequences.

In like manner, the introducing and measuring can involve five different taste receptor proteins, such as:

a polypeptide that comprises a polypeptide sequence of SEQ ID NO: 1, a functional fragment thereof, or a polypeptide whose sequence is at least 90% equivalent, or at least 95% equivalent, or at least 97% equivalent, to either of the foregoing; a polypeptide that comprises a polypeptide sequence of SEQ ID NO: 2, a functional fragment thereof, or a polypeptide whose sequence is at least 90% equivalent, or at least 95% equivalent, or at least 97% equivalent, to either of the foregoing; a polypeptide that comprises a polypeptide sequence of SEQ ID NO: 3, a functional fragment thereof, or a polypeptide whose sequence is at least 90% equivalent, or at least 95% equivalent, or at least 97% equivalent, to either of the foregoing, and a polypeptide that comprises a polypeptide sequence of SEQ ID NO: 4, a functional fragment thereof, or a polypeptide whose sequence is at least 90% equivalent, or at least 95% equivalent, or at least 97% equivalent, to either of the foregoing, and a polypeptide that comprises a polypeptide sequence of SEQ ID NO: 5, a functional fragment thereof, or a polypeptide whose sequence is at least 90% equivalent, or at least 95% equivalent, or at least 97% equivalent, to either of the foregoing; with corresponding measuring steps for each of the three different polypeptide sequences.

The foregoing embodiments involve measuring the response of each of a taste receptor protein to the test compound by comparing the activity of the taste receptor proteins to the bitter compound in the presence and the absence of the test compound. This measuring can be carried out by any suitable means. For example, in some embodiments, each taste receptor protein is expressed on the surface of a cell, and compositions comprising the bitter taste compound (both in the presence and absence of the test compound) are screened against the cells expressing a taste receptor protein in a standard cellular assay. Measuring the binding can be carried out by any suitable means typically used in determining protein binding in cellular assays. Suitable methods include, but are not limited to, use of fluorescent dyes, a calcium indicator protein, a fluorescent cAMP indicator, and the like. Activity of the test compound is determined by its ability to antagonize binding of the bitter taste compound to one or more of the taste receptor proteins.

In some further embodiments of any of the foregoing embodiments, the methods further comprise identifying an active test compound that reduces bitter taste based on the measured response. According to the foregoing embodiments, the identified active test compound is one that antagonizes the binding of the bitter compound to one, two, three, four, or five of the taste receptor proteins. For example, in some embodiments, the identified active test compound is a compound that antagonizes the binding of the bitter compound to one of the foregoing taste receptor proteins. In some other embodiments, the identified active test compound is a compound that antagonizes the binding of the bitter compound to two of the foregoing different combinations of taste receptor proteins. In some other embodiments, the identified active test compound is a compound that antagonizes the binding of the bitter compound to three of the foregoing different combinations of taste receptor proteins.

In some embodiments of any of the foregoing embodiments, the test compound is determined to be an active compound because it antagonizes at least 30%, or at least 40%, or at least 50%, or at least 60%, or at least 70%, or at least 80%, or at least 90%, of the binding activity of the bitter compound.

In some further embodiments, the methods further comprise selecting the active test compound as a compound that reduces bitter taste. The selecting can be carried out by any suitable means once the active test compounds are identified.

The test compound can be any suitable compound that is amenable for use in cellular screening assays. For example, in some embodiments, the test compound is an organic compound. In some embodiments, the test compound is a naturally occurring compound or a glycosylated derivative thereof. In some other embodiments, the test compound is a non-naturally occurring compound.

Any suitable composition can be used to provide the bitter compound. In some embodiments, the bitter compound is an extract of a plant from the Meliaceae family or a plant from the Rutacaea family. In some further embodiments, the bitter compound is an extract of a plant from the Rutacaea family and the Citrus genus, such as an extract from the tree or fruit of orange, lemon, grapefruit, lime, kumquat, pomelo, tangelo, ugli, tangerine, or yuzu. In some further embodiments, the bitter compound is a triterpene. In some embodiments, the bitter compound is a tetranortriterpenoid. In some embodiments, the bitter compound is a limonoid. In some embodiments, the bitter compound is limonin, nomilin, nomilic acid, azadirachtin, or any combination thereof. In some further embodiments, the bitter compound is limonin.

### Uses and Methods

In other aspects, the disclosure provides uses of any identified or selected active compounds of the foregoing aspects, including any embodiments or combination of embodiments thereof, as set forth above. In certain related aspects, the disclosure provides uses of any identified or selected active compounds of the foregoing aspects, including any embodiments or combination of embodiments thereof, as set forth above, to enhance the sweetness of an ingestible composition. In some embodiments thereof, the ingestible composition comprises a sweetener, such as a caloric sweetener. In certain other related aspects, the disclosure provides uses of any identified or selected active compounds of the foregoing aspects, including any embodiments or combination of embodiments thereof, as set forth above, to reduce the sourness of an ingestible composition. In some embodiments, the identified or selected active compounds are polymethoxyflavones (PMFs), such as PMFs derived from Citrus fruits. In some such embodiments, the PMFs are selected from the group consisting of sinensetin, 4′-hydroxy-sinensetin, quercetogetin, nobiletin, tangeretin, heptamethoxyflavone, and any combinations thereof.

In certain related aspects, the disclosure provides uses of polymethoxyflavones (PMFs), such as PMFs derived from Citrus fruits, to reduce the bitterness of one or more bitter compounds. In some embodiments thereof, the one or more bitter compounds are comprised by an ingestible composition. In some embodiments thereof, the ingestible composition is a composition derived from Citrus fruit. In some embodiments, the ingestible composition is a naturally occurring composition. In some other embodiments, the ingestible composition is a non-naturally occurring composition. In some such embodiments, the PMFs are selected from the group consisting of sinensetin, 4-hydroxy-sinensetin, quercetogetin, nobiletin, tangeretin, heptamethoxyflavone, and any combinations thereof.

The bitter compounds can be any suitable bitter compounds commonly found in natural food products. In some embodiments, the bitter compounds are compounds that modulates (e.g., activates) a polypeptide sequence of SEQ ID NO: 2, or a functional fragment thereof, or a polypeptide sequence whose sequence is at least 90% equivalent, or at least 95% equivalent, or at least 97% equivalent, to either of the foregoing.

In some embodiments, the bitter compound are compounds extracted of a plant from the Meliaceae family or a plant from the Rutacaea family. In some further embodiments, the bitter compound is an extract of a plant from the Rutacaea family and the Citrus genus, such as an extract from the tree or fruit of orange, lemon, grapefruit, lime, kumquat, pomelo, tangelo, ugli, tangerine, or yuzu. In some embodiments, the bitter compound is a triterpene. In some further embodiments, the bitter compound is a tetranortriterpenoid. In some further embodiments, the bitter compound is a limonoid. In some such embodiments, the bitter compound is limonin, nomilin, nomilic acid, azadirachtin, or any combination thereof. In some embodiments, the bitter compound is limonin. In some embodiments, the bitter compound is nomilin.

The disclosure also provides methods that correspond to each of the foregoing uses. Thus, in certain related aspects, the disclosure provides methods of enhancing the sweetness of an ingestible composition, comprising introducing an amount (such as a sweetness-enhancing effective amount) of any identified or selected active compounds of foregoing aspects, including any embodiments or combination of embodiments thereof, as set forth above, to the ingestible composition. In some other related aspects, the disclosure provides methods of reducing the bitterness of an ingestible composition, comprising introducing an amount (such as a bitterness-reducing effective amount) of any identified or selected active compounds of foregoing aspects, including any embodiments or combination of embodiments thereof, as set forth above, to the ingestible composition. In some embodiments, the identified or selected active compounds are polymethoxyflavones (PMFs), such as PMFs derived from Citrus fruits. In some such embodiments, the PMFs are selected from the group consisting of sinensetin, 4′-hydroxy-sinensetin, quercetogetin, nobiletin, tangeretin, heptamethoxyflavone, and any combinations thereof.

In certain related aspects, the disclosure provides methods of reducing the bitterness of an ingestible composition, comprising introducing an amount (such as a bitterness-reducing effective amount) of polymethoxyflavones (PMFs) to an ingestible composition. In some embodiments thereof, the ingestible composition comprises one or more bitter compounds. In some embodiments thereof, the ingestible composition is a composition derived from Citrus fruit. In some embodiments, the ingestible composition is a naturally occurring composition. In some other embodiments, the ingestible composition is a non-naturally occurring composition. In some such embodiments, the PMFs are selected from the group consisting of sinensetin, 4′-hydroxy-sinensetin, quercetogetin, nobiletin, tangeretin, heptamethoxyflavone, and any combinations thereof.

The bitter compounds can be any suitable bitter compounds commonly found in natural food products. In some embodiments, the bitter compounds are compounds that modulates (e.g., activates) a polypeptide sequence of SEQ ID NO: 2, or a functional fragment thereof, or a polypeptide sequence whose sequence is at least 90% equivalent, or at least 95% equivalent, or at least 97% equivalent, to either of the foregoing.

In some embodiments, the bitter compound are compounds extracted of a plant from the Meliaceae family or a plant from the Rutacaea family. In some further embodiments, the bitter compound is an extract of a plant from the Rutacaea family and the Citrus genus, such as an extract from the tree or fruit of orange, lemon, grapefruit, lime, kumquat, pomelo, tangelo, ugli, tangerine, or yuzu. In some embodiments, the bitter compound is a triterpene. In some further embodiments, the bitter compound is a tetranortriterpenoid. In some further embodiments, the bitter compound is a limonoid. In some such embodiments, the bitter compound is limonin, nomilin, nomilic acid, azadirachtin, or any combination thereof. In some embodiments, the bitter compound is limonin. In some embodiments, the bitter compound is nomilin.

The foregoing uses and methods generally involve the use of the identified or selected active compounds or PMFs in a composition containing one or more additional ingredients. For example, in at least one aspect, the disclosure provides compositions comprising any identified or selected active compounds of the foregoing aspects, including any embodiments or combination of embodiments thereof, as set forth above, wherein the identified or selected active compounds make up at least 50% by weight of the compositions on a dry weight basis (e.g., based on the total weight of the composition excluding the weight of any liquid carrier). In a related aspect, the disclosure provides solid-state compositions comprising any identified or selected active compounds of the foregoing aspects, including any embodiments or combination of embodiments thereof, as set forth above, wherein the identified or selected active compounds make up at least 50% by weight of the solid-state compositions, based on the total weight of composition. In another related aspect, the disclosure provides ingestible compositions comprising identified or selected active compounds of the foregoing aspects, including any embodiments or combination of embodiments thereof, as set forth above, wherein the concentration of the identified or selected active compounds in the ingestible compositions is no more than 200 ppm. In another related aspect, the disclosure provides ingestible compositions comprising any identified or selected active compounds of the foregoing aspects, including any embodiments or combination of embodiments thereof, as set forth above, wherein the ingestible compositions comprise no more than 1000 ppm, or no more than 900 ppm, or no more than 800 ppm, or no more than 700 ppm, or no more than 600 ppm, or no more than 500 ppm, or no more than 400 ppm, or no more than 300 ppm, or no more than 200 ppm, or no more than 100 ppm, or no more than 50 ppm, of steviol glycosides (including rebaudioside A). In another related aspect, the disclosure provides ingestible compositions comprising any identified or selected active compounds of the foregoing aspects, including any embodiments or combination of embodiments thereof, as set forth above, wherein the ingestible compositions comprise a caloric sweetener, such as sucrose, fructose, xylitol, erythritol, or combinations thereof. In another related aspect, the disclosure provides a concentrated sweetening composition comprising any flavor-modifying compounds of the foregoing aspects, including any embodiments or combination of embodiments thereof, as set forth above, and a sweetener.

In certain embodiments of any aspects and embodiments set forth herein that refer to an ingestible composition, the ingestible composition is a non-naturally-occurring product, such as a composition specifically manufactured for the production of a flavored product, such as food or beverage product.

In certain aspects, the disclosure provides ingestible compositions comprising one or more polymethoxyflavones (PMFs). In some embodiments, the one or more PMFs are present in the ingestible composition in a bitterness-reducing effective amount. In some embodiments thereof, the ingestible composition comprises one or more bitter compounds. In some embodiments thereof, the ingestible composition is a composition derived from Citrus fruit. In some embodiments, the ingestible composition is a naturally occurring composition. In some other embodiments, the ingestible composition is a non-naturally occurring composition. In some such embodiments, the PMFs are selected from the group consisting of sinensetin, 4′-hydroxy-sinensetin, quercetogetin, nobiletin, tangeretin, heptamethoxyflavone, and any combinations thereof.

The bitter compounds can be any suitable bitter compounds commonly found in natural food products. In some embodiments, the bitter compounds are compounds that modulates (e.g., activates) a polypeptide sequence of SEQ ID NO: 2, or a functional fragment thereof, or a polypeptide sequence whose sequence is at least 90% equivalent, or at least 95% equivalent, or at least 97% equivalent, to either of the foregoing.

In some embodiments, the bitter compound are compounds extracted of a plant from the Meliaceae family or a plant from the Rutacaea family. In some further embodiments, the bitter compound is an extract of a plant from the Rutacaea family and the Citrus genus, such as an extract from the tree or fruit of orange, lemon, grapefruit, lime, kumquat, pomelo, tangelo, ugli, tangerine, or yuzu. In some embodiments, the bitter compound is a triterpene. In some further embodiments, the bitter compound is a tetranortriterpenoid. In some further embodiments, the bitter compound is a limonoid. In some such embodiments, the bitter compound is limonin, nomilin, nomilic acid, azadirachtin, or any combination thereof. In some embodiments, the bitter compound is limonin. In some embodiments, the bitter compound is nomilin.

In the preceding aspect, or in any other aspect that refers to an ingestible composition, the PMFs can be present in the composition in any suitable concentration. For example, in some embodiments, the PMFs are present in the ingestible composition at a concentration ranging from 1 ppm to 1000 ppm, or from 1 ppm to 900 ppm, or from 1 ppm to 800 ppm, or from 1 ppm to 700 ppm, or from 1 ppm to 600 ppm, or from 1 ppm to 500 ppm, or from 1 ppm to 400 ppm, or from 1 ppm to 300 ppm, or from 1 ppm to 250 ppm, or from 1 ppm to 200 ppm, or from 1 ppm to 150 ppm, or from 1 ppm to 100 ppm, or from 1 ppm to 80 ppm, or from 1 ppm to 70 ppm, or from 1 ppm to 60 ppm, or from 1 ppm to 50 ppm, or from 1 ppm to 40 ppm, or from 1 ppm to 30 ppm, or from 1 ppm to 20 ppm, or from 5 ppm to 1000 ppm, or from 5 ppm to 900 ppm, or from 5 ppm to 800 ppm, or from 5 ppm to 700 ppm, or from 5 ppm to 600 ppm, or from 5 ppm to 500 ppm, or from 5 ppm to 400 ppm, or from 5 ppm to 300 ppm, or from 5 ppm to 250 ppm, or from 5 ppm to 200 ppm, or from 5 ppm to 150 ppm, or from 5 ppm to 100 ppm, or from 5 ppm to 80 ppm, or from 5 ppm to 70 ppm, or from 5 ppm to 60 ppm, or from 5 ppm to 50 ppm, or from 5 ppm to 40 ppm, or from 5 ppm to 30 ppm, or from 5 ppm to 20 ppm. In some embodiments, at least a portion (e.g., at least 10% by weight, or at least 20% by weight, or at least 30% by weight) of the PMFs is introduced to the ingestible composition, e.g., such that the concentration of PMFs is higher than what would be present in an extract of a natural product.

In general, compounds as disclosed and described herein, individually or in combination, can be provided in a composition, such as an ingestible composition. In one embodiment, compounds as disclosed and described herein, individually or in combination, can impart a more sugar-like temporal profile or flavor profile to a sweetener composition by combining one or more of the compounds as disclosed and described herein with one or more sweeteners in the sweetener composition. In another embodiment, compounds as disclosed and described herein, individually or in combination, can increase or enhance the sweet taste of a composition by contacting the composition thereof with the compounds as disclosed and described herein to form a modified composition.

Thus, in some embodiments, the compositions set forth in any of the foregoing aspects (including in any uses or methods), comprise an identified or selected active compound and a sweetener. In some embodiments, the composition further comprises a vehicle. In some embodiments, the vehicle is water. In some embodiments, the identified or selected active compound is present at a concentration at or below its sweetness recognition threshold.

For example, in some embodiments, the sweetener is present in an amount from about 0.1% to about 12% by weight. In some embodiments, the sweetener is present in an amount from about 0.2% to about 10% by weight. In some embodiments, the sweetener is present in an amount from about 0.3% to about 8% by weight. In some embodiments, the sweetener is present in an amount from about 0.4% to about 6% by weight. In some embodiments, the sweetener is present in an amount from about 0.5% to about 5% by weight. In some embodiments, the sweetener is present in an amount from about 1% to about 2% by weight. In some embodiments, the sweetener is present in an amount from about 0.1% to about 5% by weight. In some embodiments, the sweetener is present in an amount from about 0.1% to about 4% by weight. In some embodiments, the sweetener is present in an amount from about 0.1% to about 3% by weight. In some embodiments, the sweetener is present in an amount from about 0.1% to about 2% by weight. In some embodiments, the sweetener is present in an amount from about 0.1% to about 1% by weight. In some embodiments, the sweetener is present in an amount from about 0.1% to about 0.5% by weight. In some embodiments, the sweetener is present in an amount from about 0.5% to about 10% by weight. In some embodiments, the sweetener is present in an amount from about 2% to about 8% by weight. In some further embodiments of the embodiments set forth in this paragraph, the sweetener is sucrose, fructose, glucose, xylitol, erythritol, or combinations thereof.

In some other embodiments, the sweetener is present in an amount from 10 ppm to 1000 ppm. In some embodiments, the sweetener is present in an amount from 20 ppm to 800 ppm. In some embodiments, the sweetener is present in an amount from 30 ppm to 600 ppm. In some embodiments, the sweetener is present in an amount from 40 ppm to 500 ppm. In some embodiments, the sweetener is present in an amount from 50 ppm to 400 ppm. In some embodiments, the sweetener is present in an amount from 50 ppm to 300 ppm. In some embodiments, the sweetener is present in an amount from 50 ppm to 200 ppm. In some embodiments, the sweetener is present in an amount from 50 ppm to 150 ppm. In some further embodiments of the embodiments set forth in this paragraph, the sweetener is a steviol glycoside, a mogroside, a derivative of either of the foregoing, such as glycoside derivatives (e.g., glucosylates), or any combination thereof. In some embodiments, the sweetener is a steviol glycoside, such as rebaudioside A, rebaudioside M, rebaudioside D, rebaudioside B, or any combination thereof. In some embodiments, the sweetener is a mogroside, such as mogroside V, isomogroside V, isomogroside IV_{E}, mogroside IIIE, siamenoside I, the alpha isomer of siamenoside I, or any combination thereof. In some embodiments, the sweetener is sucrose, fructose, glucose, allulose, erythritol, or any combination thereof.

The compositions can include any suitable sweeteners or combination of sweeteners. In some embodiments, the sweetener is a common saccharide sweeteners, such as sucrose, fructose, glucose, and sweetener compositions comprising natural sugars, such as corn syrup (including high fructose corn syrup) or other syrups or sweetener concentrates derived from natural fruit and vegetable sources. In some embodiments, the sweetener is sucrose, fructose, or a combination thereof. In some embodiments, the sweetener is sucrose. In some other embodiments, the sweetener is selected from rare natural sugars including D-allose, D-psicose, L-ribose, D-tagatose, L-glucose, L-fucose, L-arbinose, D-turanose, and D-leucrose. In some embodiments, the sweetener is selected from semi-synthetic “sugar alcohol” sweeteners such as erythritol, isomalt, lactitol, mannitol, sorbitol, xylitol, maltodextrin, and the like. In some embodiments, the sweetener is selected from artificial sweeteners such as aspartame, saccharin, acesulfame-K, cyclamate, sucralose, and alitame. In some embodiments, the sweetener is selected from the group consisting of cyclamic acid, mogroside, tagatose, maltose, galactose, mannose, sucrose, fructose, lactose, neotame and other aspartame derivatives, glucose, D-tryptophan, glycine, maltitol, lactitol, isomalt, hydrogenated glucose syrup (HGS), hydrogenated starch hydrolyzate (HSH), stevioside, rebaudioside A, other sweet Stevia-based glycosides, chemically modified steviol glycosides (such as glucosylated steviol glycosides), mogrosides, chemically modified mogrosides (such as glucosylated mogrosides), carrelame and other guanidine-based sweeteners. In some embodiments, the sweetener is a combination of two or more of the sweeteners set forth in this paragraph. In some embodiments, the sweetener may combinations of two, three, four or five sweeteners as disclosed herein. In some embodiments, the sweetener may be a sugar. In some embodiments, the sweetener may be a combination of one or more sugars and other natural and artificial sweeteners. In some embodiments, the sweetener is a sugar. In some embodiments, the sugar is cane sugar. In some embodiments, the sugar is beet sugar. In some embodiments, the sugar may be sucrose, fructose, glucose or combinations thereof. In some embodiments, the sugar may be sucrose. In some embodiments, the sugar may be a combination of fructose and glucose.

The sweetener can also include, for example, sweetener compositions comprising one or more natural or synthetic carbohydrate, such as corn syrup, high fructose corn syrup, high maltose corn syrup, glucose syrup, sucralose syrup, hydrogenated glucose syrup (HGS), hydrogenated starch hydrolyzate (HSH), or other syrups or sweetener concentrates derived from natural fruit and vegetable sources, or semi-synthetic “sugar alcohol” sweeteners such as polyols. Non-limiting examples of polyols in some embodiments include erythritol, maltitol, mannitol, sorbitol, lactitol, xylitol, isomalt, propylene glycol, glycerol (glycerin), threitol, galactitol, palatinose, reduced isomalto-oligosaccharides, reduced xylo-oligosaccharides, reduced gentio-oligosaccharides, reduced maltose syrup, reduced glucose syrup, isomaltulose, maltodextrin, and the like, and sugar alcohols or any other carbohydrates or combinations thereof capable of being reduced which do not adversely affect taste.

The sweetener may be a natural or synthetic sweetener that includes, but is not limited to, agave inulin, agave nectar, agave syrup, amazake, brazzein, brown rice syrup, coconut crystals, coconut sugars, coconut syrup, date sugar, fructans (also referred to as inulin fiber, fructo-oligosaccharides, or oligo-fructose), green Stevia powder, Stevia rebaudiana, rebaudioside A, rebaudioside B, rebaudioside C, rebaudioside D, rebaudioside E, rebaudioside F, rebaudioside I, rebaudioside H, rebaudioside L, rebaudioside K, rebaudioside J, rebaudioside N, rebaudioside O, rebaudioside M and other sweet Stevia-based glycosides, stevioside, stevioside extracts, honey, Jerusalem artichoke syrup, licorice root, luo han guo (fruit, powder, or extracts), lucuma (fruit, powder, or extracts), maple sap (including, for example, sap extracted from Acer saccharum, Acer nigrum, Acer rubrum, Acer saccharinum, Acer platanoides, Acer negundo, Acer macrophyllum, Acer grandidentatum, Acer glabrum, Acer mono), maple syrup, maple sugar, walnut sap (including, for example, sap extracted from Juglans cinerea, Juglans nigra, Juglans ailatifolia, Juglans regia), birch sap (including, for example, sap extracted from Betula papyrifera, Betula alleghaniensis, Betula lenta, Betula nigra, Betula populifolia, Betula pendula), sycamore sap (such as, for example, sap extracted from Platanus occidentalis), ironwood sap (such as, for example, sap extracted from Ostrya virginiana), mascobado, molasses (such as, for example, blackstrap molasses), molasses sugar, monatin, monellin, cane sugar (also referred to as natural sugar, unrefined cane sugar, or sucrose), palm sugar, panocha, piloncillo, rapadura, raw sugar, rice syrup, sorghum, sorghum syrup, cassava syrup (also referred to as tapioca syrup), thaumatin, yacon root, malt syrup, barley malt syrup, barley malt powder, beet sugar, cane sugar, crystalline juice crystals, caramel, carbitol, carob syrup, castor sugar, hydrogenated starch hydrolates, hydrolyzed can juice, hydrolyzed starch, invert sugar, anethole, arabinogalactan, arrope, syrup, P-4000, acesulfame potassium (also referred to as acesulfame K or ace-K), alitame (also referred to as aclame), advantame, aspartame, baiyunoside, neotame, benzamide derivatives, bernadame, canderel, carrelame and other guanidine-based sweeteners, vegetable fiber, corn sugar, coupling sugars, curculin, cyclamates, cyclocarioside I, demerara, dextran, dextrin, diastatic malt, dulcin, sucrol, valzin, dulcoside A, dulcoside B, emulin, enoxolone, maltodextrin, saccharin, estragole, ethyl maltol, glucin, gluconic acid, glucono-lactone, glucosamine, glucoronic acid, glycerol, glycine, glycyphillin, glycyrrhizin, glycyrrhetic acid monoglucuronide, golden sugar, yellow sugar, golden syrup, granulated sugar, gynostemma, hernandulcin, isomerized liquid sugars, jallab, chicory root dietary fiber, kynurenine derivatives (including N′-formyl-kynurenine, N′-acetyl-kynurenine, 6-chloro-kynurenine), galactitol, litesse, ligicane, lycasin, lugduname, guanidine, falernum, mabinlin I, mabinlin II, maltol, maltisorb, maltodextrin, maltotriol, mannosamine, miraculin, mizuame, mogrosides (including, for example, mogroside IV, mogroside V, and neomogroside), mukurozioside, nano sugar, naringin dihydrochalcone, neohesperidine dihydrochalcone, nib sugar, nigero-oligosaccharide, norbu, orgeat syrup, osladin, pekmez, pentadin, periandrin I, perillaldehyde, perillartine, petphyllum, phenylalanine, phlomisoside I, phlorodizin, phyllodulcin, polyglycitol syrups, polypodoside A, pterocaryoside A, pterocaryoside B, rebiana, refiners syrup, rub syrup, rubusoside, selligueain A, shugr, siamenoside I, siraitia grosvenorii, soybean oligosaccharide, Splenda, SRI oxime V, steviol glycoside, steviolbioside, stevioside, strogins 1, 2, and 4, sucronic acid, sucrononate, sugar, suosan, phloridzin, superaspartame, tetrasaccharide, threitol, treacle, trilobtain, tryptophan and derivatives (6-trifluoromethyl-tryptophan, 6-chloro-D-tryptophan), vanilla sugar, volemitol, birch syrup, aspartame-acesulfame, assugrin, and combinations or blends of any two or more thereof.

In still other embodiments, the sweetener can be a chemically or enzymatically modified natural high potency sweetener. Modified natural high potency sweeteners include glycosylated natural high potency sweetener such as glucosyl-, galactosyl-, or fructosyl-derivatives containing 1-50 glycosidic residues. Glycosylated natural high potency sweeteners may be prepared by enzymatic transglycosylation reaction catalyzed by various enzymes possessing transglycosylating activity. In some embodiments, the modified sweetener can be substituted or unsubstituted.

Additional sweeteners also include combinations of any two or more of any of the aforementioned sweeteners. In some embodiments, the sweetener may comprise combinations of two, three, four or five sweeteners as disclosed herein. In some embodiments, the sweetener may be a sugar. In some embodiments, the sweetener may be a combination of one or more sugars and other natural and artificial sweeteners. In some embodiments, the sweetener is a caloric sweetener, such as sucrose, fructose, xylitol, erythritol, or combinations thereof. In some embodiments, the ingestible compositions are free (or, in some embodiments) substantially free of Stevia-derived sweeteners, such as steviol glycosides, glucosylated steviol glycosides, or rebaudiosides. For example, in some embodiments, the ingestible compositions are either free of Stevia-derived sweeteners or comprise Stevia-derived sweeteners in a concentration of no more than 1000 ppm, or no more than 500 ppm, or no more than 200 ppm, or no more than 100 ppm, or no more than 50 ppm, or no more than 20 ppm, or no more than 10 ppm, or no more than 5 ppm, or no more than 3 ppm, or no more than 1 ppm.

The identified or selected active compounds can be present in the ingestible compositions in any suitable amount. In some embodiments, the identified or selected active compounds are present in an amount sufficient to enhance the taste (e.g., enhance the sweetness, reduce the sourness, or reduce the bitterness) of the compositions. Thus, in some embodiments, the ingestible composition comprises the identified or selected active compound in a concentration no greater than 200 ppm, or no greater than 150 ppm, or no greater than 100 ppm, or no greater than 50 ppm, or no greater than 40 ppm, or no greater than 30 ppm, or no greater than 20 ppm. In some embodiments, the identified or selected active compound is present in a minimum amount, such as 1 ppm or 5 ppm. Thus, in some embodiments, the ingestible composition comprises the identified or selected active compound in a concentration ranging from 1 ppm to 200 ppm, or from 1 ppm to 150 ppm, or from 1 ppm to 100 ppm, or from 1 ppm to 50 ppm, or from 1 ppm to 40 ppm, or from 1 ppm to 30 ppm, or from 1 ppm to 20 ppm, or from 5 ppm to 200 ppm, or from 5 ppm to 150 ppm, or from 5 ppm to 100 ppm, or from 5 ppm to 50 ppm, or from 5 ppm to 40 ppm, or from 5 ppm to 30 ppm, or from 5 ppm to 20 ppm. In embodiments where a sweetener, such as sucrose or fructose, are present, the weight-to-weight ratio of sweetener to the identified or selected active compound in the ingestible composition ranges from 1000:1 to 50000:1, or from 1000:1 to 10000:1, or from 2000:1 to 8000:1.

The ingestible compositions or sweetener concentrates can, in certain embodiments, comprise any additional ingredients or combination of ingredients as are commonly used in food and beverage products, including, but not limited to:

acids, including, for example citric acid, phosphoric acid, ascorbic acid, sodium acid sulfate, lactic acid, or tartaric acid;

bitter ingredients, including, for example caffeine, quinine, green tea, catechins, polyphenols, green robusta coffee extract, green coffee extract, potassium chloride, menthol, or proteins (such as proteins and protein isolates derived from plants, algae, or fungi);

coloring agents, including, for example caramel color, Red #40, Yellow #5, Yellow #6, Blue #1, Red #3, purple carrot, black carrot juice, purple sweet potato, vegetable juice, fruit juice, beta carotene, turmeric curcumin, or titanium dioxide;

preservatives, including, for example sodium benzoate, potassium benzoate, potassium sorbate, sodium metabisulfate, sorbic acid, or benzoic acid;

antioxidants including, for example ascorbic acid, calcium disodium EDTA, alpha tocopherols, mixed tocopherols, rosemary extract, grape seed extract, resveratrol, or sodium hexametaphosphate;

vitamins or functional ingredients including, for example resveratrol, Co-Q10, omega 3 fatty acids, theanine, choline chloride (citocoline), fibersol, inulin (chicory root), taurine, Panax ginseng extract, guanana extract, ginger extract, L-phenylalanine, L-carnitine, L-tartrate, D-glucoronolactone, inositol, bioflavonoids, Echinacea, ginko biloba, yerba mate, flax seed oil, garcinia cambogia rind extract, white tea extract, ribose, milk thistle extract, grape seed extract, pyrodixine HCl (vitamin B6), cyanoobalamin (vitamin B12), niacinamide (vitamin B3), biotin, calcium lactate, calcium pantothenate (pantothenic acid), calcium phosphate, calcium carbonate, chromium chloride, chromium polynicotinate, cupric sulfate, folic acid, ferric pyrophosphate, iron, magnesium lactate, magnesium carbonate, magnesium sulfate, monopotassium phosphate, monosodium phosphate, phosphorus, potassium iodide, potassium phosphate, riboflavin, sodium sulfate, sodium gluconate, sodium polyphosphate, sodium bicarbonate, thiamine mononitrate, vitamin D3, vitamin A palmitate, zinc gluconate, zinc lactate, or zinc sulphate;

clouding agents, including, for example ester gun, brominated vegetable oil (BVO), or sucrose acetate isobutyrate (SAIB);

buffers, including, for example sodium citrate, potassium citrate, or salt;

flavors, including, for example propylene glycol, ethyl alcohol, glycerine, gum Arabic (gum acacia), maltodextrin, modified corn starch, dextrose, natural flavor, natural flavor with other natural flavors (natural flavor WONF), natural and artificial flavors, artificial flavor, silicon dioxide, magnesium carbonate, or tricalcium phosphate; or

starches and stabilizers, including, for example pectin, xanthan gum, carboxylmethylcellulose (CMC), polysorbate 60, polysorbate 80, medium chain triglycerides, cellulose gel, cellulose gum, sodium caseinate, modified food starch, gum Arabic (gum acacia), inulin, or carrageenan.

The ingestible compositions or sweetener concentrates can have any suitable pH. In some embodiments, the identified or selected active compounds enhance the sweetness of a sweetener under a broad range of pH, e.g., from lower pH to neutral pH. The lower and neutral pH includes, but is not limited to, a pH from about 2.5 to about 8.5; from about 3.0 to about 8.0; from about 3.5 to about 7.5; and from about 4.0 to about 7. In certain embodiments, compounds as disclosed and described herein, individually or in combination, can enhance the perceived sweetness of a fixed concentration of a sweetener in taste tests at a compound concentration of about 50 μM, 40 μM, 30 μM, 20 μM, or 10 μM at both low to neutral pH value. In certain embodiments, the enhancement factor of the compounds as disclosed and described herein, individually or in combination, at the lower pH is substantially similar to the enhancement factor of the compounds at neutral pH. Such consistent sweet enhancing property under a broad range of pH allow a broad use in a wide variety of foods and beverages of the compounds as disclosed and described herein, individually or in combination.

The ingestible compositions set forth according to any of the foregoing embodiments, also include, in certain embodiments, one or more additional identified or selected active compounds, such as compounds that enhance sweetness (e.g., hesperetin, naringenin, glucosylated steviol glycosides, etc.), compounds that block bitterness, compounds that enhance umami, compounds that reduce sourness or licorice taste, compounds that enhance saltiness, compounds that enhance a cooling effect, or any combinations of the foregoing.

### Flavored Products and Concentrates

In certain aspects, the disclosure provides flavored products comprising any compositions of the preceding aspects. In some embodiment, the flavored products are beverage products, such as soda, flavored water, tea, and the like. In some other embodiments, the flavored products are food products, such as yogurt.

In embodiments where the flavored product is a beverage, the beverage may be selected from the group consisting of enhanced sparkling beverages, colas, lemon-lime flavored sparkling beverages, orange flavored sparkling beverages, grape flavored sparkling beverages, strawberry flavored sparkling beverages, pineapple flavored sparkling beverages, ginger-ales, root beers, fruit juices, fruit-flavored juices, juice drinks, nectars, vegetable juices, vegetable-flavored juices, sports drinks, energy drinks, enhanced water drinks, enhanced water with vitamins, near water drinks, coconut waters, tea type drinks, coffees, cocoa drinks, beverages containing milk components, beverages containing cereal extracts and smoothies. In some embodiments, the beverage may be a soft drink.

In certain embodiments of any aspects and embodiments set forth herein that refer to an flavored product, the flavored product is a non-naturally-occurring product, such as a packaged food or beverage product.

Further non-limiting examples of food and beverage products or formulations include sweet coatings, frostings, or glazes for such products or any entity included in the Soup category, the Dried Processed Food category, the Beverage category, the Ready Meal category, the Canned or Preserved Food category, the Frozen Processed Food category, the Chilled Processed Food category, the Snack Food category, the Baked Goods category, the Confectionery category, the Dairy Product category, the Ice Cream category, the Meal Replacement category, the Pasta and Noodle category, and the Sauces, Dressings, Condiments category, the Baby Food category, and/or the Spreads category.

In general, the Soup category refers to canned/preserved, dehydrated, instant, chilled, UHT and frozen soup. For the purpose of this definition soup(s) means a food prepared from meat, poultry, fish, vegetables, grains, fruit and other ingredients, cooked in a liquid which may include visible pieces of some or all of these ingredients. It may be clear (as a broth) or thick (as a chowder), smooth, pureed or chunky, ready-to-serve, semi-condensed or condensed and may be served hot or cold, as a first course or as the main course of a meal or as a between meal snack (sipped like a beverage). Soup may be used as an ingredient for preparing other meal components and may range from broths (consomme) to sauces (cream or cheese-based soups).

The Dehydrated and Culinary Food Category usually means: (i) Cooking aid products such as: powders, granules, pastes, concentrated liquid products, including concentrated bouillon, bouillon and bouillon like products in pressed cubes, tablets or powder or granulated form, which are sold separately as a finished product or as an ingredient within a product, sauces and recipe mixes (regardless of technology); (ii) Meal solutions products such as: dehydrated and freeze dried soups, including dehydrated soup mixes, dehydrated instant soups, dehydrated ready-to-cook soups, dehydrated or ambient preparations of ready-made dishes, meals and single serve entrees including pasta, potato and rice dishes; and (iii) Meal embellishment products such as: condiments, marinades, salad dressings, salad toppings, dips, breading, batter mixes, shelf stable spreads, barbecue sauces, liquid recipe mixes, concentrates, sauces or sauce mixes, including recipe mixes for salad, sold as a finished product or as an ingredient within a product, whether dehydrated, liquid or frozen.

The Beverage category usually means beverages, beverage mixes and concentrates, including but not limited to, carbonated and non-carbonated beverages, alcoholic and non-alcoholic beverages, ready to drink beverages, liquid concentrate formulations for preparing beverages such as sodas, and dry powdered beverage precursor mixes. The Beverage category also includes the alcoholic drinks, the soft drinks, sports drinks, isotonic beverages, and hot drinks. The alcoholic drinks include, but are not limited to beer, cider/perry, FABs, wine, and spirits. The soft drinks include, but are not limited to carbonates, such as colas and non-cola carbonates; fruit juice, such as juice, nectars, juice drinks and fruit flavored drinks; bottled water, which includes sparkling water, spring water and purified/table water; functional drinks, which can be carbonated or still and include sport, energy or elixir drinks; concentrates, such as liquid and powder concentrates in ready to drink measure. The drinks, either hot or cold, include, but are not limited to coffee or ice coffee, such as fresh, instant, and combined coffee; tea or ice tea, such as black, green, white, oolong, and flavored tea; and other drinks including flavor-, malt- or plant-based powders, granules, blocks or tablets mixed with milk or water.

The Snack Food category generally refers to any food that can be a light informal meal including, but not limited to Sweet and savory snacks and snack bars. Examples of snack food include, but are not limited to fruit snacks, chips/crisps, extruded snacks, tortilla/corn chips, popcorn, pretzels, nuts and other sweet and savory snacks. Examples of snack bars include, but are not limited to granola/muesli bars, breakfast bars, energy bars, fruit bars and other snack bars.

The Baked Goods category generally refers to any edible product the process of preparing which involves exposure to heat or excessive sunlight. Examples of baked goods include, but are not limited to bread, buns, cookies, muffins, cereal, toaster pastries, pastries, waffles, tortillas, biscuits, pies, bagels, tarts, quiches, cake, any baked foods, and any combination thereof.

The Ice Cream category generally refers to frozen dessert containing cream and sugar and flavoring. Examples of ice cream include, but are not limited to: impulse ice cream; take-home ice cream; frozen yoghurt and artisanal ice cream; soy, oat, bean (e.g., red bean and mung bean), and rice-based ice creams.

The Confectionery category generally refers to edible product that is sweet to the taste. Examples of confectionery include, but are not limited to candies, gelatins, chocolate confectionery, sugar confectionery, gum, and the likes and any combination products.

The Meal Replacement category generally refers to any food intended to replace the normal meals, particularly for people having health or fitness concerns. Examples of meal replacement include, but are not limited to slimming products and convalescence products.

The Ready Meal category generally refers to any food that can be served as meal without extensive preparation or processing. The ready meal includes products that have had recipe “skills” added to them by the manufacturer, resulting in a high degree of readiness, completion and convenience. Examples of ready meal include, but are not limited to canned/preserved, frozen, dried, chilled ready meals; dinner mixes; frozen pizza; chilled pizza; and prepared salads.

The Pasta and Noodle category includes any pastas and/or noodles including, but not limited to canned, dried and chilled/fresh pasta; and plain, instant, chilled, frozen and snack noodles.

The Canned/Preserved Food category includes, but is not limited to canned/preserved meat and meat products, fish/seafood, vegetables, tomatoes, beans, fruit, ready meals, soup, pasta, and other canned/preserved foods.

The Frozen Processed Food category includes, but is not limited to frozen processed red meat, processed poultry, processed fish/seafood, processed vegetables, meat substitutes, processed potatoes, bakery products, desserts, ready meals, pizza, soup, noodles, and other frozen food.

The Dried Processed Food category includes, but is not limited to rice, dessert mixes, dried ready meals, dehydrated soup, instant soup, dried pasta, plain noodles, and instant noodles. The Chill Processed Food category includes, but is not limited to chilled processed meats, processed fish/seafood products, lunch kits, fresh cut fruits, ready meals, pizza, prepared salads, soup, fresh pasta and noodles.

The Sauces, Dressings and Condiments category includes, but is not limited to tomato pastes and purees, bouillon/stock cubes, herbs and spices, monosodium glutamate (MSG), table sauces, soy based sauces, pasta sauces, wet/cooking sauces, dry sauces/powder mixes, ketchup, mayonnaise, mustard, salad dressings, vinaigrettes, dips, pickled products, and other sauces, dressings and condiments.

The Baby Food category includes, but is not limited to milk- or soybean-based formula; and prepared, dried and other baby food.

The Spreads category includes, but is not limited to jams and preserves, honey, chocolate spreads, nut based spreads, and yeast based spreads.

The Dairy Product category generally refers to edible product produced from mammal's milk. Examples of dairy product include, but are not limited to drinking milk products, cheese, yoghurt and sour milk drinks, and other dairy products.

Additional examples for flavored products, particularly food and beverage products or formulations, are provided as follows. Exemplary ingestible compositions include one or more confectioneries, chocolate confectionery, tablets, countlines, bagged selflines/softlines, boxed assortments, standard boxed assortments, twist wrapped miniatures, seasonal chocolate, chocolate with toys, alfajores, other chocolate confectionery, mints, standard mints, power mints, boiled sweets, pastilles, gums, jellies and chews, toffees, caramels and nougat, medicated confectionery, lollipops, liquorice, other sugar confectionery, bread, packaged/industrial bread, unpackaged/artisanal bread, pastries, cakes, packaged/industrial cakes, unpackaged/artisanal cakes, cookies, chocolate coated biscuits, sandwich biscuits, filled biscuits, savory biscuits and crackers, bread substitutes, breakfast cereals, rte cereals, family breakfast cereals, flakes, muesli, other cereals, children's breakfast cereals, hot cereals, ice cream, impulse ice cream, single portion dairy ice cream, single portion water ice cream, multi-pack dairy ice cream, multi-pack water ice cream, take-home ice cream, take-home dairy ice cream, ice cream desserts, bulk ice cream, take-home water ice cream, frozen yoghurt, artisanal ice cream, dairy products, milk, fresh/pasteurized milk, full fat fresh/pasteurized milk, semi skimmed fresh/pasteurized milk, long-life/uht milk, full fat long life/uht milk, semi skimmed long life/uht milk, fat-free long life/uht milk, goat milk, condensed/evaporated milk, plain condensed/evaporated milk, flavored, functional and other condensed milk, flavored milk drinks, dairy only flavored milk drinks, flavored milk drinks with fruit juice, soy milk, sour milk drinks, fermented dairy drinks, coffee whiteners, powder milk, flavored powder milk drinks, cream, cheese, processed cheese, spreadable processed cheese, unspreadable processed cheese, unprocessed cheese, spreadable unprocessed cheese, hard cheese, packaged hard cheese, unpackaged hard cheese, yoghurt, plain/natural yoghurt, flavored yoghurt, fruited yoghurt, probiotic yoghurt, drinking yoghurt, regular drinking yoghurt, probiotic drinking yoghurt, chilled and shelf-stable desserts, dairy-based desserts, soy-based desserts, chilled snacks, fromage frais and quark, plain fromage frais and quark, flavored fromage frais and quark, savory fromage frais and quark, sweet and savory snacks, fruit snacks, chips/crisps, extruded snacks, tortilla/corn chips, popcorn, pretzels, nuts, other sweet and savory snacks, snack bars, granola bars, breakfast bars, energy bars, fruit bars, other snack bars, meal replacement products, slimming products, convalescence drinks, ready meals, canned ready meals, frozen ready meals, dried ready meals, chilled ready meals, dinner mixes, frozen pizza, chilled pizza, soup, canned soup, dehydrated soup, instant soup, chilled soup, hot soup, frozen soup, pasta, canned pasta, dried pasta, chilled/fresh pasta, noodles, plain noodles, instant noodles, cups/bowl instant noodles, pouch instant noodles, chilled noodles, snack noodles, canned food, canned meat and meat products, canned fish/seafood, canned vegetables, canned tomatoes, canned beans, canned fruit, canned ready meals, canned soup, canned pasta, other canned foods, frozen food, frozen processed red meat, frozen processed poultry, frozen processed fish/seafood, frozen processed vegetables, frozen meat substitutes, frozen potatoes, oven baked potato chips, other oven baked potato products, non-oven frozen potatoes, frozen bakery products, frozen desserts, frozen ready meals, frozen pizza, frozen soup, frozen noodles, other frozen food, dried food, dessert mixes, dried ready meals, dehydrated soup, instant soup, dried pasta, plain noodles, instant noodles, cups/bowl instant noodles, pouch instant noodles, chilled food, chilled processed meats, chilled fish/seafood products, chilled processed fish, chilled coated fish, chilled smoked fish, chilled lunch kit, chilled ready meals, chilled pizza, chilled soup, chilled/fresh pasta, chilled noodles, oils and fats, olive oil, vegetable and seed oil, cooking fats, butter, margarine, spreadable oils and fats, functional spreadable oils and fats, sauces, dressings and condiments, tomato pastes and purees, bouillon/stock cubes, stock cubes, gravy granules, liquid stocks and fonds, herbs and spices, fermented sauces, soy based sauces, pasta sauces, wet sauces, dry sauces/powder mixes, ketchup, mayonnaise, regular mayonnaise, mustard, salad dressings, regular salad dressings, low fat salad dressings, vinaigrettes, dips, pickled products, other sauces, dressings and condiments, baby food, milk formula, standard milk formula, follow-on milk formula, toddler milk formula, hypoallergenic milk formula, prepared baby food, dried baby food, other baby food, spreads, jams and preserves, honey, chocolate spreads, nut-based spreads, and yeast-based spreads. Exemplary ingestible compositions also include confectioneries, bakery products, ice creams, dairy products, sweet and savory snacks, snack bars, meal replacement products, ready meals, soups, pastas, noodles, canned foods, frozen foods, dried foods, chilled foods, oils and fats, baby foods, or spreads or a mixture thereof. Exemplary ingestible compositions also include breakfast cereals, sweet beverages or solid or liquid concentrate compositions for preparing beverages, ideally so as to enable the reduction in concentration of previously known saccharide sweeteners, or artificial sweeteners.

Some embodiments provide a chewable composition that may or may not be intended to be swallowed. In some embodiments, the chewable composition may be gum, chewing gum, sugarized gum, sugar-free gum, functional gum, bubble gum including compounds as disclosed and described herein, individually or in combination.

Typically at least a sweet receptor modulating amount, a sweet receptor ligand modulating amount, a sweet flavor modulating amount, a sweet flavoring agent amount, a sweet flavor enhancing amount, or a therapeutically effective amount of one or more of the present compounds will be added to the ingestible composition, optionally in the presence of sweeteners so that the sweet flavor modified ingestible composition has an increased sweet taste as compared to the ingestible composition prepared without the compounds of the present invention, as judged by human beings or animals in general, or in the case of formulations testing, as judged by a majority of a panel of at least eight human taste testers, via procedures commonly known in the field.

In some embodiments, compounds as disclosed and described herein, individually or in combination, modulate the sweet taste or other taste properties of other natural or synthetic sweet tastants, and ingestible compositions made therefrom. In one embodiment, the compounds as disclosed and described herein, individually or in combination, may be used or provided in its ligand enhancing concentration(s). For example, the compounds as disclosed and described herein, individually or in combination, may be present in an amount of from 0.001 ppm to 100 ppm, or narrower alternative ranges from 0.1 ppm to 50 ppm, from 0.01 ppm to 40 ppm, from 0.05 ppm to 30 ppm, from 0.01 ppm to 25 ppm, or from 0.1 ppm to 30 ppm, or from 0.1 ppm to 25 ppm, or from 1 ppm to 30 ppm, or from 1 ppm to 25 ppm.

In some embodiments, identified or selected active compounds as disclosed and described herein, individually or in combination, may be provided in a flavoring concentrate formulation, e.g., suitable for subsequent processing to produce a ready-to-use (i.e., ready-to-serve) product. By “a flavoring concentrate formulation”, it is meant a formulation which should be reconstituted with one or more diluting medium to become a ready-to-use composition. The term “ready-to-use composition” is used herein interchangeably with “ingestible composition”, which denotes any substance that, either alone or together with another substance, can be taken by mouth whether intended for consumption or not. In one embodiment, the ready-to-use composition includes a composition that can be directly consumed by a human or animal. The flavoring concentrate formulation is typically used by mixing with or diluted by one or more diluting medium, e.g., any consumable or ingestible ingredient or product, to impart or modify one or more flavors to the diluting medium. Such a use process is often referred to as reconstitution. The reconstitution can be conducted in a household setting or an industrial setting. For example, a frozen fruit juice concentrate can be reconstituted with water or other aqueous medium by a consumer in a kitchen to obtain the ready-to-use fruit juice beverage. In another example, a soft drink syrup concentrate can be reconstituted with water or other aqueous medium by a manufacturer in large industrial scales to produce the ready-to-use soft drinks. Since the flavoring concentrate formulation has the flavoring agent or flavor modifying agent in a concentration higher than the ready-to-use composition, the flavoring concentrate formulation is typically not suitable for being consumed directly without reconstitution. There are many benefits of using and producing a flavoring concentrate formulation. For example, one benefit is the reduction in weight and volume for transportation as the flavoring concentrate formulation can be reconstituted at the time of usage by the addition of suitable solvent, solid or liquid.

The flavored products set forth according to any of the foregoing embodiments, also include, in certain embodiments, one or more additional flavor-modifying compounds, such as compounds that enhance sweetness (e.g., hesperetin, naringenin, glucosylated steviol glycosides, etc.), compounds that block bitterness, compounds that enhance umami, compounds that reduce sourness, compounds that enhance saltiness, compounds that enhance a cooling effect, or any combinations of the foregoing.

In certain embodiments of any aspects and embodiments set forth herein that refer to a sweetening or flavoring concentrate, the sweetening or flavoring concentrate is a non-naturally-occurring product, such as a composition specifically manufactured for the production of a flavored product, such as food or beverage product.

In one embodiment, the flavoring concentrate formulation comprises i) compounds as disclosed and described herein, individually or in combination; ii) a carrier; and iii) optionally at least one adjuvant. The term “carrier” denotes a usually inactive accessory substance, such as solvents, binders, or other inert medium, which is used in combination with the present compound and one or more optional adjuvants to form the formulation. For example, water or starch can be a carrier for a flavoring concentrate formulation. In some embodiments, the carrier is the same as the diluting medium for reconstituting the flavoring concentrate formulation; and in other embodiments, the carrier is different from the diluting medium. The term “carrier” as used herein includes, but is not limited to, ingestibly acceptable carrier.

The term “adjuvant” denotes an additive which supplements, stabilizes, maintains, or enhances the intended function or effectiveness of the active ingredient, such as the compound of the present invention. In one embodiment, the at least one adjuvant comprises one or more flavoring agents. The flavoring agent may be of any flavor known to one skilled in the art or consumers, such as the flavor of chocolate, coffee, tea, mocha, French vanilla, peanut butter, chai, or combinations thereof. In another embodiment, the at least one adjuvant comprises one or more sweeteners. The one or more sweeteners can be any of the sweeteners described in this application. In another embodiment, the at least one adjuvant comprises one or more ingredients selected from the group consisting of a emulsifier, a stabilizer, an antimicrobial preservative, an antioxidant, vitamins, minerals, fats, starches, protein concentrates and isolates, salts, and combinations thereof. Examples of emulsifiers, stabilizers, antimicrobial preservatives, antioxidants, vitamins, minerals, fats, starches, protein concentrates and isolates, and salts are described in U.S. Pat. No. 6,468,576, the content of which is hereby incorporated by reference in its entirety for all purposes.

In one embodiment, the present flavoring concentrate formulation can be in a form selected from the group consisting of liquid including solution and suspension, solid, foamy material, paste, gel, cream, and a combination thereof, such as a liquid containing certain amount of solid contents. In one embodiment, the flavoring concentrate formulation is in form of a liquid including aqueous-based and nonaqueous-based. In some embodiments, the present flavoring concentrate formulation can be carbonated or non-carbonated.

The flavoring concentrate formulation may further comprise a freezing point depressant, nucleating agent, or both as the at least one adjuvant. The freezing point depressant is an ingestibly acceptable compound or agent which can depress the freezing point of a liquid or solvent to which the compound or agent is added. That is, a liquid or solution containing the freezing point depressant has a lower freezing point than the liquid or solvent without the freezing point depressant. In addition to depress the onset freezing point, the freezing point depressant may also lower the water activity of the flavoring concentrate formulation. The examples of the freezing point depressant include, but are not limited to, carbohydrates, oils, ethyl alcohol, polyol, e.g., glycerol, and combinations thereof. The nucleating agent denotes an ingestibly acceptable compound or agent which is able to facilitate nucleation. The presence of nucleating agent in the flavoring concentrate formulation can improve the mouthfeel of the frozen Blushes of a frozen slush and to help maintain the physical properties and performance of the slush at freezing temperatures by increasing the number of desirable ice crystallization centers. Examples of nucleating agents include, but are not limited to, calcium silicate, calcium carbonate, titanium dioxide, and combinations thereof.

In one embodiment, the flavoring concentrate formulation is formulated to have a low water activity for extended shelf life. Water activity is the ratio of the vapor pressure of water in a formulation to the vapor pressure of pure water at the same temperature. In one embodiment, the flavoring concentrate formulation has a water activity of less than about 0.85. In another embodiment, the flavoring concentrate formulation has a water activity of less than about 0.80. In another embodiment, the flavoring concentrate formulation has a water activity of less than about 0.75.

In one embodiment, the flavoring concentrate formulation has the present compound in a concentration that is at least 2 times of the concentration of the compound in a ready-to-use composition. In one embodiment, the flavoring concentrate formulation has the present compound in a concentration that is at least 5 times of the concentration of the compound in a ready-to-use composition. In one embodiment, the flavoring concentrate formulation has the present compound in a concentration that is at least 10 times of the concentration of the compound in a ready-to-use composition. In one embodiment, the flavoring concentrate formulation has the present compound in a concentration that is at least 15 times of the concentration of the compound in a ready-to-use composition. In one embodiment, the flavoring concentrate formulation has the present compound in a concentration that is at least 20 times of the concentration of the compound in a ready-to-use composition. In one embodiment, the flavoring concentrate formulation has the present compound in a concentration that is at least 30 times of the concentration of the compound in a ready-to-use composition. In one embodiment, the flavoring concentrate formulation has the present compound in a concentration that is at least 40 times of the concentration of the compound in a ready-to-use composition. In one embodiment, the flavoring concentrate formulation has the present compound in a concentration that is at least 50 times of the concentration of the compound in a ready-to-use composition. In one embodiment, the flavoring concentrate formulation has the present compound in a concentration that is at least 60 times of the concentration of the compound in a ready-to-use composition. In one embodiment, the flavoring concentrate formulation has the present compound in a concentration that is up to 100 times of the concentration of the compound in a ready-to-use composition.

The sweetening or flavoring concentrates set forth according to any of the foregoing embodiments, also include, in certain embodiments, one or more additional flavor-modifying compounds, such as compounds that enhance sweetness (e.g., hesperetin, naringenin, glucosylated steviol glycosides, etc.), compounds that block bitterness (e.g., eriodictyol, homoeriodictyol, sterubin, and salts or glycoside derivatives thereof, as well as vanillyl lignans, e.g., matairesinol and other compounds set forth in PCT Publication No. WO 2012/146584), compounds that enhance umami (e.g., rubemamine, rubescenamine, (E)-3-(3,4-dimethoxyphenyl)-N-(4-methoxyphenethyl)acrylamide, and the like), compounds that reduce sourness and/or licorice taste, compounds that enhance saltiness, compounds that enhance a cooling effect, or any combinations of the foregoing.

### Treatment of Compositions of Diseased Citrus

In a fifteenth aspect, the disclosure provides methods for reducing the bitterness of Citrus compositions derived from Citrus fruit having Citrus greening disease, the method comprising: (a) deriving a Citrus composition from Citrus fruit, wherein at least a portion of the Citrus fruit has Citrus greening disease; and (b) introducing to the Citrus composition a bitterness-reducing composition, which comprises polymethoxyflavones (PMFs), such as an amount (such as a bitterness-reducing effective amount) of polymethoxyflavones (PMFs).

In some embodiments, the Citrus composition comprises an abnormally elevated concentration of one or more bitter compounds, such as abnormally elevated levels of limonin or nomilin. In some embodiments, the bitter compounds are compounds that modulates (e.g., activates) a polypeptide sequence of SEQ ID NO: 2, or a functional fragment thereof, or a polypeptide sequence whose sequence is at least 90% equivalent, or at least 95% equivalent, or at least 97% equivalent, to either of the foregoing. In some embodiments, the bitter compounds are limonoids, such as limonin, nomilin, nomilinic acid, or any combination thereof.

Any suitable PMFs can be used. In some embodiments, the PMFs are selected from the group consisting of sinensetin, 4′-hydroxy-sinensetin, quercetogetin, nobiletin, tangeretin, heptamethoxyflavone, and any combinations thereof. In some embodiments, the bitterness-reducing composition comprising the PMFs is derived from Citrus waste.

### Blocking Bitterness in Pharmaceutical APIs

Many drug compounds impart a bitter taste, which therefore limits the ways in which they can be formulated and administered. Therefore, in certain aspects, the disclosure provides a pharmaceutical composition comprising a bitter-tasting pharmaceutical active ingredient and one or more polymethoxyflavones (PMFs). In some embodiments, the one or more PMFs are selected from the group consisting of” sinensetin, 4′-hydroxy-sinensetin, quercetogetin, nobiletin, tangeretin, heptamethoxyflavone, and any combinations thereof. Such pharmaceutical compositions can be in any suitable form for oral administration, such as tablets, lozenges, capsules, powders, liquid solutions, liquid suspensions, and the like. Such pharmaceutical compositions can include any suitable pharmaceutical excipients, binders, and the like, such as those set forth in Remington's Pharmaceutical Sciences. In some embodiments, the bitter-tasting pharmaceutical active ingredient is an ion channel inhibitor, such as a proton channel inhibitor. Other examples of bitter-tasting APIs whose bitterness is reduced by the one or more PMFs include, but are not limited to, atropine, brinzolamide, chloramphenicol, chloroquine, clindamycin, dexamethasone, digoxin, diltiazem, diphenhydramine, docusate, dorzolamide, doxepin, doxylamine, enalapril, erythromycin, esomeprazole, famotidine, gabapentin, ginkgolide A, guaifenesin, L-histidine, lomefloxacin, methylprednisolone, ofloxacin, oleuropein, oxyphenonium, pirenzepine, prednisone, ranitidine, trapidil, trimethoprim, and cetirizine.

### EXAMPLES

To further illustrate this invention, the following examples are included. The examples should not, of course, be construed as specifically limiting the invention. Variations of these examples within the scope of the claims are within the purview of one skilled in the art and are considered to fall within the scope of the invention as described, and claimed herein. The reader will recognize that the skilled artisan, armed with the present disclosure, and skill in the art is able to prepare and use the invention without exhaustive examples.

### Example 1—Taste Receptor Identification

Cells expressing a promiscuous G protein (G16g44) were transfected using TransIT-293 (Mirus) with expression vectors encoding 22 functional biter receptor cDNAs, with one receptor per transfection. The cells were transfected directly in 384-well format high-density plates and incubated for 28 hours. Later, cells were loaded with the Ca-specific dye Fluo4-AM. After an incubation of 60 minutes, excess dye is washed off, and cells are stimulated with respective bitter agonists while simultaneously recording changes in intracellular fluorescence using a FLIPR instrument. Bitter agonist and responding receptors were identified and respective stable bitter receptor cell lines were then used in the same assay to perform full dose-response analysis with the respective bitter agonists.

The above protocol was carried out using limonin as a non-limiting example of a bitter agonist. When using limonin, a positive dose response was recorded in the concentration range of 12.5 μM to 50 μM for five different bitter receptor proteins expressed in cells. The five expressed receptor proteins correspond to the amino acid sequences identified herein as SEQ ID NO: 1, 2, 3, 4, and 5.

### Example 2—Bitterness Antagonism

Certain PMFs were tested for their ability to antagonize binding of limonin to cells expressing the polypeptide identified herein as SEQ ID NO: 2. Table 1 shows the percent activity of limonin with the polypeptide at a concentration of 25 μM both in the absence of a test antagonist and in the presence of a test antagonist at concentration ranging from 50 μM to 200 μM.

TABLE 1

| Antagonist | Conc. (μM) | % Limonin Activation |
|---|---|---|
| None | — | 98 |
| 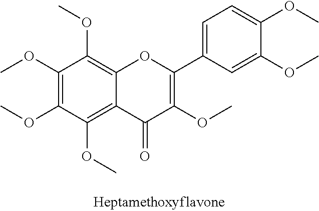 | 200 | 79 |
| 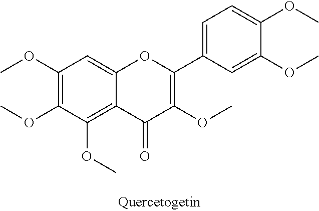 | 100 | 77 |
| 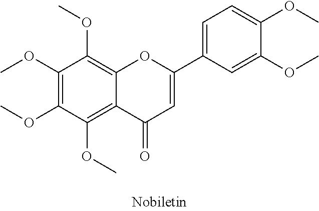 | 200 | 74 |
| 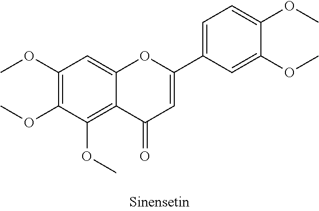 | 200 | 70 |
| 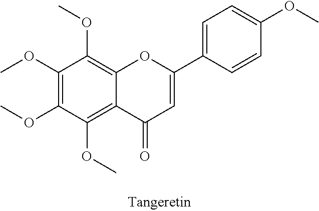 | 50 | 76 |
| 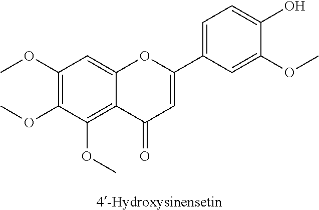 | 200 | 64 |

1. A method of identifying compounds that reduce bitter taste, the method comprising:
introducing a test compound and a bitter compound to one or more taste receptor proteins, wherein the one or more taste receptor proteins are polypeptides that comprise: a polypeptide sequence of SEQ ID NO: 1, or a functional fragment thereof; a polypeptide sequence of SEQ ID NO: 2, or a functional fragment thereof; a polypeptide sequence of SEQ ID NO: 3, or a functional fragment thereof; a polypeptide sequence of SEQ ID NO: 4, or a functional fragment thereof; a polypeptide sequence of SEQ ID NO: 5, or a functional fragment thereof; or a polypeptide sequence whose sequence is at least 90% equivalent to any of the foregoing; and
measuring a response of each of the one or more taste receptor proteins to the test compound by comparing an activity of the one or more taste receptor proteins to the bitter compound in the presence and the absence of the test compound.

2. The method of claim 1,
wherein the introducing step comprises introducing a test compound and a bitter compound to two or more taste receptor proteins, wherein the two or more taste receptor proteins are independently polypeptides that comprise: a polypeptide sequence of SEQ ID NO: 2, or a functional fragment thereof; a polypeptide sequence of SEQ ID NO: 4, or a functional fragment thereof; a polypeptide sequence of SEQ ID NO: 5, or a functional fragment thereof; or a polypeptide sequence whose sequence is at least 90% equivalent to any of the foregoing; and
wherein the measuring step comprises measuring a response of each of the two or more taste receptor proteins to the test compound by comparing an activity of the one or more taste receptor proteins to the bitter compound in the presence and the absence of the test compound.

3. The method of claim 1 or 2, further comprising:
identifying an active test compound that reduces bitter taste based on the measured response.

4. The method of claim 3, further comprising:
selecting the active test compound as a compound that reduces bitter taste.

5. The method of any one of claims 1 to 4, wherein the test compound is a naturally occurring compound or a glycosylated derivative thereof.

6. The method of any one of claims 1 to 4, wherein the test compound is a non-naturally occurring compound.

7. The method of any one of claims 1 to 6, wherein the bitter compound is an extract of a plant from the Meliaceae family or a plant from the Rutacaea family.

8. The method of claim 7, wherein the bitter compound is an extract of a plant from the Rutacaea family and the Citrus genus, such as an extract from the tree or fruit of orange, lemon, grapefruit, lime, kumquat, pomelo, tangelo, ugli, tangerine, or yuzu.

9. The method of any one of claims 1 to 8, wherein the bitter compound is a triterpene.

10. The method of claim 9, wherein the bitter compound is a tetranortriterpenoid.

11. The method of any one of claims 1 to 10, wherein the bitter compound is a limonoid.

12. The method of claim 11, wherein the bitter compound is limonin, nomilin, nomilic acid, azadirachtin, or any combination thereof.

13. The method of claim 12, wherein the bitter compound is limonin.

14. The method of any one of claims 1 to 13, wherein the one or more taste receptor proteins are each expressed on a surface of a cell.

15. The method of claim 14, wherein the cell is a eukaryotic cell.
